SEQUENCE LISTING (13 sequences)

SEQ ID NO: 1 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKASKQQTNIDVIYERVRDDYQLHWTSTNWKGTNTKDKWIDRSSERYKIDWEKEEMTN

SEQ ID NO: 2 - protein, 319 aa, Staphylococcus aureus
MKTRIVSSVTTTLLLGSILMNPVANAADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 3 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAAENFLDPNKASSLLSSGFSPDFATVITMDRKASKQQTNIDVIYERVRDDYQLHWTSTNWKGTNTKDKWIDRSSERYKIDWEKEEMTN

SEQ ID NO: 4 - protein, 293 aa, Staphylococcus aureus
ADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 5 - DNA, 601 bp, Staphylococcus aureus
atgaaaacacgtatagtcagctcagtaacaacaacactattgctaggttccatattaatgattcctgtcgtcaatgccgcagattctgatattaatattaaaaccggtactacagatattggaagcattactacagtaaaaacaggtgatttagtcacttatgataaagaaaatggcatgcacaaaaaagtattttatagttttatcgatgataaaaatcacaataaaaaactgctagttattagaacgaaaggtaccattgctggtcaatatagaggttatagcgaagaaggtgctaacaaaagtggtttagccyggccttcagcctttaaggtacagttgcaactacctgataatgaagtagctcaaatatctgattactatccagagaattcgattgatacaaaagagtatatgagtactttaacttatggattcaacggtaaatgttactggtgatgatacaggaaaaattggcggccttattggtgcaaatgtttcgattggtcatacactgaaatatgttcaacctgatttcaaaacaattttagagagcccaactgataaaaaagtaggctggaaagtgatatttaacaatatg

SEQ ID NO: 6 - protein, 5 aa, Artificial Sequence
  Sythesized
GPGPG

SEQ ID NO: 7 - protein, 270 aa, Artificial Sequence
  Synthesized
ADSDINIKTGTTDIGSNTTVKTGDLVTYDKENGMHKKVFYSFIDDKNHNKGPGPGADSDINIKTGTTDIGSNTTVKTGDLVTYDKENGMHKKVFYSFIDDKNHNKGPGPGADSDINIKTGTTDIGSNTTVKTGDLVTYDKENGMHKKVFYSFIDDKNHNKGPGPGADSDINIKTGTTDIGSNTTVKTGDLVTYDKENGMHKKVFYSFIDDKNHNKGPGPGADSDINIKTGTTDIGSNTTVKTGDLVTYDKENGMHKKVFYSFIDDKNHNK

SEQ ID NO: 8 - protein, 95 aa, Artificial Sequence
  Synthesized
KKVFYSFIDDKNHNKGPGPGKKVFYSFIDDKNHNKGPGPGKKVFYSFIDDKNHNKGPGPGKKVFYSFIDDKNHNKGPGPGKKVFYSFIDDKNHNK

SEQ ID NO: 9 - protein, 95 aa, Artificial Sequence
  Synthesized
KKVFYSFIDDKNHNKGPGPGKLLVIRTKGTIAGQYGPGPGTDKKVGWKVIFNNMVGPGPGGWKVIFNNMVNQNWGGPGPGYGNQLFMKTRNGSMK

SEQ ID NO: 10 - protein, 55 aa, Artificial Sequence
  Synthesized
KKVFYSFIDDKNHNKGPGPGTDKKVGWKVIFNNMVGPGPGKLLVIRTKGTIAGQY

SEQ ID NO: 11 - protein, 95 aa, Artificial Sequence
  Synthesized
TDKKVGWKVIFNNMVGPGPGTDKKVGWKVIFNNMVGPGPGTDKKVGWKVIFNNMVGPGPGTDKKVGWKVIFNNMVGPGPGTDKKVGWKVIFNNMV

SEQ ID NO: 12 - DNA, 105 bp, Artificial Sequence
  Synthesized
tcgcatatgaaaacacgtatagtcagctcagtaacaacaacactattgctaggttccatattaatgaatcctgtcgctaatgccgcagatcaagcacgtgaatta

SEQ ID NO: 13 - DNA, 30 bp, Artificial Sequence
  Synthesized
tctggatccttatggtgaaacacaacgacc

(19) United States
(12) Patent Application Publication
(10) Pub. No.: US 2023/0295246 A1
(43) Pub. Date: Sep. 21, 2023

(54) COMPOSITIONS AND METHODS FOR IMMUNIZATION AGAINST STAPHYLOCOCCUS AUREUS

(71) Applicant: Washington University, St. Louis, MO (US)

(72) Inventors: Juliane Bubeck Wardenburg, St. Louis, MO (US); Brandon Lee, St. Louis, MO (US); Reuben Olaniyi, St. Louis, MO (US)

(21) Appl. No.: 17/787,091

(22) PCT Filed: Dec. 21, 2020
(86) PCT No.: PCT/US2020/066463

§ 371 Date: Jun. 17, 2022

Related U.S. Application Data
(60) Provisional application No. 62/951,829, filed on Dec. 20, 2019.

(51) Int. Cl. C07K 14/31; A61P 31/04

(52) U.S. Cl. CPC C07K 14/31; A61P 31/04; A61K 2039/55

(57) ABSTRACT

Among the various aspects of the present disclosure relates to methods and compositions useful to reduce or prevent tolerogenic or suppressive T-cell responses in a subject to a bacterial pathogen. The methods include exposing the subject to an modified bacterial antigen prior to the first exposure of the subject to the bacterial pathogen. The vaccination strategy is capable of neutralizing Hla to provide immunoprotection against S. aureus infections.

### CROSS-REFERENCE TO RELATED APPLICATION

This application claims benefit from U.S. Provisional Application Ser. No. 62/951,829 filed on Dec. 20, 2019, which is incorporated herein by reference in its entirety.

### GOVERNMENTAL RIGHTS

This disclosure was made with government support under A1097434 awarded by the National Institutes of Health. The government has certain rights in the disclosure.

### FIELD OF THE TECHNOLOGY

The present disclosure relates generally to the fields of immunology, microbiology, infectious diseases and medicine. Specifically, the present disclosure relates to methods and compositions including an exotoxin protein, such as α-hemolysin, for producing an immune response to a bacterium.

### BACKGROUND

Staphylococcus aureus is both a human skin commensal and a leading cause of infection. Skin and soft tissue infection (SSTI) remains the most common form of S. aureus disease with an incidence of >100 cases per 100,000, costing >$4 billion/year in the U.S. SST's can lead to disseminated disease, and have exacerbated the health burden of antibiotic-resistance. Efforts to develop vaccines against S. aureus have failed, and correlates of human immunity remain elusive.

Recurrence of SSTI can exceed 50%, primarily afflicting those at the extremes of age and individuals with certain underlying diseases including immunodeficiency and diabetes. In contrast, <20% of patients who recover from invasive disease experience reinfection. This dichotomy suggests that staphylococcal immunity depends on the initial infection site, and may relate to temporal determinants of exposure. While the molecular pathogenesis of recurrent infection is poorly understood, host and pathogen factors contribute to susceptibility. Humans harboring defects in neutrophil and T cell function and IL-17 signaling present with recurrent infection, corroborated by mouse models that demonstrate the importance of innate and adaptive immunity. S. aureus thwarts immunity through an array of virulence factors including α-toxin (Hla), a pore-forming cytotoxin that contributes to superficial and invasive disease, perturbing host immunity during skin infection and recurrent disease. Commensurate with this observation, the anti-Hla antibody response is a correlate of protection against recurrent skin infection and bacteremia.

There remains a need in the art for additional compositions and methods for preventing and/or treating pathogenic infections (e.g., staphylococcal infections), as well as the attenuation or amelioration of the secondary effects of such an infection.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1S show primary S. aureus skin infection blunts the development of immunity and tissue-specific patterning of immunity against secondary S. aureus skin infection. FIG. 1A shows the experimental timeline of primary intravenous (5×10⁶ CFU/mouse) and skin infections (1×10⁸ CFU/mouse) and skin reinfection (1×10⁸ CFU/mouse). FIG. 1B shows representative gross and histopathologic (H&E-stained) images of skin lesions on post infection day 4. Arrows denote dermonecrosis. Scale bars: 1000 μm. FIG. 1A shows the quantitation of dermonecrotic area following secondary skin infection in mice subjected to primary skin or intravenous challenge. FIG. 1D shows CFU analysis of lesions from mice in FIG. 1C. ****P<0.0001, by parametric 2-tailed Student's t test after log₁₀ transformation and confirmation of normality with Shapiro-Wilk and Anderson-Darling tests. Data are representative of 3 (FIGS. 1A-1C) and 2 (FIG. 1D) independent experiments. FIG. 1E shows weight loss in mice following primary S. aureus bacteremia. FIG. 1F shows the area of dermonecrosis quantified in mice following S. aureus skin infection. For FIG. 1E and FIG. 1F, n=8 mice, error bars represent the standard deviation. FIG. 1G shows the quantification of anti-Hla titer in mice following primary and secondary bacteremia or skin infection. FIG. 1H shows the quantification of anti-S. aureus titer in mice following primary and secondary bacteremia or skin infection. FIG. 1I shows total CD19⁺B cells in the draining lymph nodes and spleens following primary bacteremia or skin infection. FIG. 1J Gross pathologic findings in secondary skin infection in mMT and wild-type (WT) mice that were exposed to primary bacteremia. Quantification of dermonecrosis area (FIG. 1K), colony forming unit (CFU) recovery (FIG. 1L), and anti-Hla titer (FIG. 1M). N) Gross pathologic findings and (FIG. 1K) dermonecrosis area in secondary skin infection in mice following primary intravenous or skin infection with 5×10⁶ CFU. FIG. 1P shows an experimental timeline of CD4⁺ T cell depletion in mice subjected to secondary skin infection following primary bacteremia infection. FIG. 1Q shows the quantification of anti-Hla titer 35 days post-T cell depletion. FIG. 1R shows gross pathologic findings and S) dermonecrosis area in mice subjected to secondary skin infection following primary bacteremia infection; mice subjected to secondary skin infection following primary skin infection are included as a control. *=p<0.05; **=p<0.01; ***=p<0.001; ****=p<0.0001 determined by one-way ANOVA with Sidak's multiple comparison test, or t test where appropriate. Data are representative of (FIG. 1J-1L) two or (FIG. 1P-1S) three independent experiments.

FIG. 2A-2N show antigen-specific T cell priming depends on tissue site of infection. FIG. 2A shows the experimental timeline of infection with S. aureus USA300_{OVA} and cellular analysis. FIG. 2B shows the quantification of OT-II T cells harvested from skin dLNs and spleen 7 days after primary infection (1×108 CFU/mouse for skin infection, and 5×10⁶ CFU/mouse for bacteremia) and from pooled dLNs and spleen 14 days after infection. FIG. 2C shows OT-II T cells as in FIG. 2B, classified into EM, CM, and naive phenotypes. FIG. 2D shows the analysis of IFN-γ expression by cells harvested from infected mice. FIG. 2E shows the timeline of infection and cellular analysis following secondary infection. FIG. 2F shows OVA-specific T cell quantification from pooled dLNs and spleens following secondary infection in mice as in FIG. 2E shows each dot represents 1 independent group of least 5 mice. *P<0.05 and **P<0.01, by 1-way ANOVA with Sidak's multiple comparisons test (FIG. 2B and FIG. 2C) or parametric 2-tailed Student's t test (FIG. 2D). Data are representative of 3 independent experiments (FIG. 2A-FIG. 2D). FIG. 2F shows depiction of OVA expression constructs pww412OVA and pKLOVA for use in S. aureus. pKLOVA includes an improved translation initiation region, containing a translational enhancer (ENH) from gene 10 of phage T7 and optimized Shine-Dalgarno (SD) sequence downstream of the Igt promoter. FIG. 2G shows a western blot analysis of OVA138-386 (top) and control Hla (bottom) expression in overnight supernatants of S. aureus strain USA₃₀₀OVA. FIG. 2H shows OVA-specific T cell cytokine analysis following primary skin or bacteremic infection. FIG. 2I shows the quantification of OVA-specific T cells from pooled dLNs and spleens following secondary infection in mice infected as in FIG. 2E. Naïve (CD44low) and memory (CD44high) OT-IIs were identified by flow cytometry. Each dot represents one independent experimental group of at least 5 mice. Quantification of FIG. 2K CD11 b⁺ DC, FIG. 2L CD103⁺ DCs, and FIG. 2M Langerhans cells in dLN and skin following primary skin infection in mice subjected to vaccination with Adjuvant or HlaH35L prior to infection with USA300. FIG. 2N shows S. aureus CFU recovery from skin lesions in mice vaccinated and infected as in I-K. *=p<0.05; **=p<0.01; ***=p<0.001; determined by parametric two-tailed t test. Data are representative of (FIG. 2F, FIG. 2G) two or (FIG. 2I-FIG. 2L) three independent experiments.

FIG. 3A-3D show Hla alters the skin DC response. FIG. 3A shows total DC accumulation in skin dLNs and skin in mice subjected to primary skin infection with USA300 or USA300 hla::erm (1×10⁸ CFU/mouse). FIG. 3B shows CD11b⁺ DC accumulation following infection. FIG. 3C shows CD103+DC accumulation following infection. FIG. 3D shows LC accumulation following infection as in FIG. 3A. *P<0.05 and **P<0.01, by parametric 2-tailed Student's t test. Data are representative of 3 independent experiments.

FIG. 4A-4I shows modulation of the antigen-specific T cell response by Hla. FIG. 4A shows OT-II T cell quantification in the dLNs and FIG. 4B shows the spleen, classified into EM, CM, and naive phenotypes 7 days after infection with USA300_{OVA} or USA300_{OVA} hla::erm (1×10⁸ CFU/mouse). FIG. 4C shows skin OT-II T cell accumulation 7 days after infection as in FIG. 4A. FIG. 4D shows DCs in the dLNs and skin in mice subjected to adjuvant only or Hla_{H35L} vaccination prior to USA300_{OVA} infection. FIG. 4E OT-II T cell quantification in the dLNs and spleen in mice subjected to adjuvant only or Hla_{H35L} vaccination prior to infection with USA300_{OVA}. FIG. 4F Quantification of EM cells from mice as in FIG. 4E. FIG. 4G shows the skin gross pathology 4 days after infection in mice born to adjuvant only or Hla_{H35L}-vaccinated dams. FIG. 4H shows the quantification of OT-II T cells in the dLNs and spleen following infection of mice with USA300_{OVA} as in FIG. 4G. FIG. 4I shows EM T cell phenotype analysis of cells harvested as in FIG. 4H. *P<0.05, **P<0.01, ***P<0.001, and ****P<0.0001, by multiple t test comparison with Bonferroni-Dunn correction (FIG. 4A and FIG. 4B), parametric 2-tailed Student's t test (FIG. 4C— FIG. 4E and FIG. 4H), or 2-way ANOVA with Sidak's multiple comparisons test (FIG. 4F and FIG. 4I). Data are representative of 2 independent experiments.

FIG. 5A-5F show T cell response modulation by Hla during infection. FIG. 5A shows a diagram illustrating experimental protocol, with single cell bzsed RNA transcript analysis performed on CD45+ cells harvested from lymphoid organs 7 days post-infection of mice with S. aureus wild-type or Hla-USA300 strains. FIG. 5B shows tsne clustering of transcripts define broad cell populations of interest including B cells and CD4⁺/CD8⁺ T cells for analysis. FIG. 5C shows a detailed evaluation of the CD4⁺ T cell compartment RNA transcript analysis, revealing distinct cell subsets and gene expression profiles that define each cell subset. Highlighted in the panel on the right are cell clusters that demonstrate a cellular phenotype indicative of antigen experience during infection, or T cell activation. FIG. 5D shows activated T cell cluster analysis, comparing wild-type infection to Hla-USA300 infection. Analysis of effector memory CD4⁺ T cells (cluster 6) and Rorc+CD4⁺ T cell populations reveals increased recovery of these cell clusters following infection with the Hla-deficient USA300 strain. FIG. 5E shows detailed evaluation of the CD8⁺ T cell compartment RNA transcript analysis, revealing distinct cell subsets and gene expression profiles that define each cell subset. Highlighted in the panel on the right are cell clusters that demonstrate a cellular phenotype indicative of antigen experience during infection, or T cell activation. FIG. 5F shows gene profiling of independent transcripts that are increased in T cells following infection with Hla-USA300 (red) or downregulated in T cells following infection with Hla-USA300 (green). CD4⁺ T cell cluster 6 is presented on the left, CD8⁺ T cell cluster 9 is presented on the right.

FIG. 6A-6B show the host response to S. aureus infection following vaccination with candidate Hla vaccine antigens and control antigens. FIG. 6A shows images of skin lesions in groups of mice vaccinated with control PBS, distinct Hla antigens, or OVA. FIG. 6A is a plot showing correlation of anti-Hla antibody titer (half-maximal titer, Log EC₅₀, defined by ELISA) to protection against red blood cell lysis (% lysis) by purified active Hla as a measure of antibody-based toxin neutralization capability.

FIG. 7 shows a model of Hla action on the T cell compartment. (upper panel) In the presence of Hla in an unvaccinated state, Hla acts on the T cell compartment to dampen the generation of antigen-specific effector T cells capable of generating protection against S. aureus infection. (lower panel) When Hla is neutralized by vaccination, its action on the T cell compartment is precluded thus enabling the generation of a diverse antigen-specific T cell response that confers protection against S. aureus infection.

### DETAILED DESCRIPTION

The present disclosure is based, at least in part, that neutralization of a bacterial antigen prior to a first infection is required to protect the host immune response, specifically the adaptive immune response. For example, neutralization of α-hemolysin (Hla) prior to the time of initial infection is required to protect the natural development of the antigen-specific T cell response. Prior vaccination efforts, for example vaccination to S. aureus, have targeted the development of anti-staphylococcal antibody responses to the immunizing antigens to: 1) augment bacterial clearance, or 2) protect against tissue injury. In contrast, the present approach is predicated on neutralizing the bacterial toxin (e.g., Hla) in order to protect the endogenous T cell response to infection. The compositions and method of the present disclosure enable the development of a diverse antigen-specific T cell repertoire following exposure to a bacterial pathogen (e.g., S. aureus).

To date, all S. aureus vaccination campaigns have targeted pre-exposed individuals that are known to possess an existing immune response against this pathogen—responses that are shaped by the effects of Hla. Vaccination of a pre-exposed population is thus expected to result in the amplification of the pre-existing, non-protective immune response. Indeed, this accounts for the failures of prior vaccine clinical trials. The present disclosure provides the rational design of vaccination at birth or shortly thereafter (prior to initial exposure to S. aureus) and a two-fold vaccine approach in which maternal immunization against Hla would elicit protection against Hla in the early weeks of life when S. aureus exposure commonly occurs, followed by active immunization of infants in the primary series to elicit active immunity against Hla. This approach is allows for the development of natural T cell-mediated immunity against S. aureus.

A composition of the disclosure may optionally comprise one or more additional drugs or therapeutically active agent in addition to an antigen as described herein. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present disclosure may themselves be provided in the form of a pharmaceutically acceptable salt), buffers, coating agents, or antioxidants.

Other aspects and iterations of the disclosure are described more thoroughly below.

### I. Compositions

(a) Antigens

The present disclosure provides compositions useful for generating an immune response in subject to a bacterial pathogen. In some embodiments, the compositions comprise a bacterial antigen for eliciting an immune response in a subject. In some embodiments, the bacterial antigen is an attenuated bacterial toxin (e.g. a toxoid). An attenuated bacterial toxin is an inactivated toxin whose toxicity has been suppressed either by mutation, chemical treatment or heat treatment, while other properties, typically immunogenicity, are maintained. Toxins are secreted by bacteria, whereas attenuated bacterial toxins are altered form of toxins; attenuated bacterial toxins are not secreted by bacteria. Thus, when used during vaccination, an immune response is mounted and immunological memory is formed against the molecular markers of the attenuated bacterial toxin without resulting in toxin-induced illness.

Staphylococcal α-hemolysin (Hla or α-toxin) is the founding member of a family of bacterial pore-forming β-barrel toxins. Its structural gene, hla, is located on the chromosome of all S. aureus strains examined that secrete the 293 residue water-soluble monomer. Studies of genetic variation in clinical isolates indicate significant Hla conservation across strains at both the nucleotide and protein level. Hla is thought to engage surface receptors of sensitive host cells, thereby promoting its oligomerization into a heptameric prepore and insertion of a β-barrel structure with 2 nm pore diameter into the plasma membrane. Hla pores form in lymphocytes, macrophages, alveolar epithelial cells, pulmonary endothelium and erythrocytes; however granulocytes and fibroblasts appear less sensitive to overt lysis. Instillation of purified Hla into rabbit or rat lung tissue triggers vascular leakage and pulmonary hypertension, which has been attributed to release of several signaling molecules, e.g. phosphatidyl inositol, nitric oxide, prostanoids (PGE2, PGI2) and thromboxane A2. In agreement with the biochemical attributes of Hla, mutations that abrogate Hla expression in S. aureus Newman severely attenuate virulence of the bacteria in the murine pneumonia model.

Monomeric Hla binds to A Disintegrin and Metalloprotease 10 (ADAM10) on the host cell surface, utilizing this protein as a toxin receptor. ADAM10 binding enables the assembly a homo-heptamer on the membrane, which is a requisite intermediate for the extension of the stem domain of the toxin through the membrane as a classic beta-barrel pore structure. Pore formation is intrinsically injurious to the host cell, however also triggers the rapid activation of ADAM10 metalloprotease activity and host tissue injury as a result of ADAM10-mediated proteolysis. While Hla is not required for S. aureus survival, this toxin is essential for pathogenesis in animal models of severe skin infection, pneumonia, sepsis, peritonitis, corneal infection, and central nervous system infection. The tissue tropism of Hla, the result of nearly ubiquitous cellular expression of ADAM10, renders this single toxin a very widely utilized virulence factor in the molecular pathogenesis of S. aureus disease.

Certain aspects of the disclosure include methods and compositions concerning compositions including polypeptides, peptides, or nucleic acid encoding a Hla protein. These proteins may be modified by deletion, insertion, and/or substitution. In particular embodiments, these proteins are capable of eliciting an immune response in a subject.

The Hla polypeptides include the amino acid sequence of Hla proteins from bacteria in the Staphylococcus genus. The Hla sequence may be from a particular staphylococcus species, such as Staphylococcus aureus, and may be from a particular strain, such as Newman. In certain embodiments, the Hla sequence can comprise a sequence having a consensus S. aureus precursor sequence of:

(SEQ ID NO: 1 and SEQ ID NO: 2)
MKTRIVSSVTTTLLLGSILMNPVANAADSDINIKTGTTDIGSNTTVKTG
DLVTYDKENGMHKKVFYSFIDDKNHNKKLLVIRTKGTIAGQYRVYSEEG
ANKSGLAWPSAFKVQLQLPDNEVAQISDYYPRNSIDTKEYMSTLTYGFN
GNVTGDDTGKIGGLIGANVSIGHTLKYVQPDFKTILESPTDKKVGWKVI
FNNMVNQNWGPYDRDSWNPVYGNQLFMKTRNGSMKAA(E/D)NFLDPNK
ASSLLSSGFSPDFATVITMDRKASKQQTNIDVIYERVRDDYQLHWTSTN
WKGTNTKDKW(I/T)DRSSERYKIDWEKEEMTN.

and a mature S. aureus consensus sequence of:

(SEQ ID NO: 3 and SEQ ID NO: 4)
ADSDINIKTGTTDIGSNTTVKTGDLVTYDKENGMHKKVFYSFIDDKNHN
KKLLVIRTKGTIAGQYRVYSEEGANKSGLAWPSAFKVQLQLPDNEVAQI
SDYYPRNSIDTKEYMSTLTYGFNGNVTGDDTGKIGGLIGANVSIGHTLK
YVQPDFKTILESPTDKKVGWKVIFNNMVNQNWGPYDRDSWNPVYGNQLF
MKTRNGSMKAA(E/D)NFLDPNKASSLLSSGFSPDFATVITMDRKASKQ
QTNIDVIYERVRDDYQLHWTSTNWKGTNTKDKW(I/T)DRSSERYKIDW
EKEEMTN.

In certain aspects, the Hla sequence is substantially set forth in Genbank Accession Numbers AAA26498 (gi152953), Mu50 (NP—371687.1) (gi15924153), COL (YP—186036.1) (gi57650272), N315 (NP—374279.1) (gi15926746), JH9 (YP—001246598.1) (gi148267655), JH1 (YP—001316387.1) (gi150393712), USA300 (YP—493756.1) (gi87160380), NCTC8325 (YP—499665.1) (gi88194865), Newman (YP—001332107.1) (gi151221285), MW2 (NP—645861.1) (gi21282773), and MSSA476 (YP—043222.1) (gi49486001), which are hereby incorporated by reference as of the earliest priority date of this application, or is a variant thereof. In further embodiments, other Hla polypeptides may be used, the sequences of which may be identified by one of skill in the art using databases and internet accessible resources.

As used herein, a “protein” or “polypeptide” refers to a molecule comprising at least ten amino acid residues. In some embodiments, wild-type versions of a protein or polypeptide are employed, however, in many embodiments of the disclosure, a modified protein or polypeptide is employed to generate an immune response. The terms described above may be used interchangeably herein. A “modified protein” or “modified polypeptide” refers to a protein or polypeptide whose chemical structure, particularly its amino acid sequence, is altered with respect to the wild-type protein or polypeptide. In some embodiments, a modified protein or polypeptide has at least one modified activity or function (recognizing that proteins or polypeptides may have multiple activities or functions). For example, a modified protein may have reduced cytotoxicity relative to the wild type protein. Thus, the modified protein can be attenuated relative to the wild type protein. It is specifically contemplated that a modified protein or polypeptide may be altered with respect to one activity or function, yet retain a wild-type activity or function in other respects, such as immunogenicity.

In some embodiments, the present disclosure provides modified Hla peptides and nucleic acids encoding the same. In one aspect, the disclosure provides modified Hla in which substitution of amino acid 35, for example, HlaH35L abrogates functional pore formation by destabilizing the heptameric structure. In some embodiments, the modified Hla is a protomer-protomer interface variant, e.g., HlaH35 variant in which histidine residue is substituted by any other amino acid. In one aspect, a modified Hla includes an amino latch (e.g., amino acids 1-20 of SEQ ID NO:1) peptide variants, including within in the context of the H35 mutant including a combination of single or multiple amino acid substitutions within the first 20 amino acids.

In some embodiments, the present disclosure provides a modified Hla comprising a substitution of amino acids 5 and 7 perturb the structure of the amino-latch, modifying the conformation of the monomer and the ability of the amino latch to contribute to stabilization of the oligomeric pore. In one aspect, the modified Hla comprises HlaI5A/I7A.

In some embodiments, the present disclosure provides a modified Hla comprising a substitution of amino acids 45 and 118 preclude the interaction of the folded prestem domain with the cap domain, thus predicted to alter the structure and receptor binding properties of the monomeric form of Hla. In one aspect, the modified Hla comprises HlaD45A/Y118F.

In some embodiments, the present disclosure provides a modified Hla comprising a substitution of amino acids 66 and 70 alters the binding properties of the toxin with the host receptor and cell membrane. In one aspect, the modified Hla comprises HlaR66A/E70A.

In some embodiments, the present disclosure provides a modified Hla comprising an Hla in which native residues Y118-V140 are replaced with an engineered peptide encompassing the predicted T cell epitope KKVFYSFIDDKNHNK (HlaK36-K50)(amino acids 1-15 of SEQ ID NO: 10) flanked by two linker sequences (GPGPG)(SEQ ID NO: 6). This variant replaces the native stem domain of Hla, the toxicity of the variant is eliminated. In one aspect, the modified Hal comprises HlaΔK110-Y148 with insertion of amino acid linker following residue 109 and Hla molecules comprising the same. In some embodiments, the present disclosure provides a modified Hla comprising a membrane insertion deletion variant, e.g., HlaΔY118-V140 with insertion of amino acid linker following residue 117 and Hla molecules comprising the same.

In some embodiments, the present disclosure provides a modified Hla comprising the amino acid sequence

(SEQ ID NO: 7)
ADSDINIKTGTTDIGSNTTVKTGDLVTYDKENGMHKKVFYSFIDDKNHN
KGPGPGADSDINIKTGTTDIGSNTTVKTGDLVTYDKENGMHKKVFYSFI
DDKNHNKGPGPGADSDINIKTGTTDIGSNTTVKTGDLVTYDKENGMHKK
VFYSFIDDKNHNKGPGPGADSDINIKTGTTDIGSNTTVKTGDLVTYDKE
NGMHKKVFYSFIDDKNHNKGPGPGADSDINIKTGTTDIGSNTTVKTGDL
VTYDKENGMHKKVFYSFIDDKNHNK.

In some embodiments, the present disclosure provides a modified Hla comprising the amino acid sequence

(SEQ ID NO: 8)
KKVFYSFIDDKNHNKGPGPGKKVFYSFIDDKNHNKGPGPGKKVFYSFID
DKNHNKGPGPGKKVFYSFIDDKNHNKGPGPGKKVFYSFIDDKNHNK.

In some embodiments, the present disclosure provides a modified Hla comprising the amino acid sequence

(SEQ ID NO: 9)
KKVFYSFIDDKNHNKGPGPGKLLVIRTKGTIAGQYGPGPGTDKKVGWKV
IFNNMVGPGPGGWKVIFNNMVNQNWGGPGPGYGNQLFMKTRNGSMK.

In some embodiments, the present disclosure provides a modified Hla comprising the amino acid sequence

(SEQ ID NO: 10)
KKVFYSFIDDKNHNKGPGPGTDKKVGWKVIFNNMVGPGPGKLLVIRTKG
TIAGQY.

In some embodiments, the present disclosure provides a modified Hla comprising the amino acid sequence

(SEQ ID NO: 11)
TDKKVGWKVIFNNMVGPGPGTDKKVGWKVIFNNMVGPGPGTDKKVGWKV
IFNNMVGPGPGTDKKVGWKVIFNNMVGPGPGTDKKVGWKVIFNNMV.

In some embodiments, the present disclosure provides a modified Hla comprising the removal of amino latch trypsin sensitivity, e.g., HlaK8A variant and Hla molecules comprising the same.

In some embodiments, the present disclosure provides a modified Hla comprising the disengagement of the pre-stem variant, predicted to expose amino latch, e.g., HlaD45A/Y118F variant and Hla molecules comprising the same.

In certain embodiments the size of an Hla protein or polypeptide (wild-type or modified) may comprise, but is not limited to, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino molecules or greater, and any range derivable therein, or derivative thereof. It is contemplated that polypeptides may be mutated by truncation, rendering them shorter than their corresponding wild-type form, but also they might be altered by fusing or conjugating a heterologous protein sequence with a particular function (e.g., for targeting or localization, for enhanced immunogenicity, for purification purposes, etc.). In some embodiments, the modified Hla according to the disclosure may be a single domain of Hla or multiple domains of Hla attached by a linker polypeptide.

As used herein, an “amino molecule” refers to any amino acid, amino acid derivative, or amino acid mimic known in the art. In certain embodiments, the residues of the proteinaceous molecule are sequential, without any non-amino molecule interrupting the sequence of amino molecule residues. In other embodiments, the sequence may comprise one or more non-amino molecule moieties. In particular embodiments, the sequence of residues of the proteinaceous molecule may be interrupted by one or more non-amino molecule moieties.

Accordingly, the term “proteinaceous composition” encompasses amino molecule sequences comprising at least one of the 20 common amino acids in naturally synthesized proteins, or at least one modified or unusual amino acid.

Proteinaceous compositions may be made by any technique known to those of skill in the art, including (i) the expression of proteins, polypeptides, or peptides through standard molecular biological techniques, (ii) the isolation of proteinaceous compounds from natural or recombinant sources (e.g., E. coli, insect cells, yeast or the like), or (iii) the chemical synthesis of proteinaceous materials. The nucleotide as well as the protein, polypeptide, and peptide sequences for various genes have been previously disclosed, and may be found in the recognized computerized databases. One such database is the National Center for Biotechnology Information's Genbank and GenPept databases. The coding regions for these genes may be amplified and/or expressed using the techniques disclosed herein or as would be known to those of ordinary skill in the art.

Amino acid sequence variants of Hla are contemplated and can be substitutional, insertional, or deletion variants. A modification in a polypeptide of the disclosure may affect 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, or more non-contiguous or contiguous amino acids of the polypeptide, as compared to wild-type. A Hla polypeptide from any staphylococcus species and strain are contemplated for use in methods of the disclosure.

Variants typically lack one or more residues of the native or wild-type protein. Individual residues can be deleted or a number of contiguous amino acids can be deleted. A stop codon may be introduced (by substitution or insertion) into an encoding nucleic acid sequence to generate a truncated protein. Insertional mutants typically involve the addition of material at a non-terminal point in the polypeptide. This may include the insertion of one or more residues. Terminal additions, called fusion proteins, may also be generated.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins of the disclosure may be recombinant, or synthesized in vitro. Alternatively, a non-recombinant or recombinant protein may be isolated from bacteria. It is also contemplated that a bacterium containing such a variant may be implemented in compositions and methods of the disclosure. Consequently, a protein need not be isolated.

The present disclosure provides recombinant polynucleotides encoding the proteins, polypeptides, peptides of the disclosure. The nucleic acid sequences for wild-type Hla, any Hla variant, or modified Hla as described herein.

As used in this application, the term “polynucleotide” refers to a nucleic acid molecule that either is recombinant or has been isolated free of total genomic nucleic acid. Included within the term “polynucleotide” are oligonucleotides (nucleic acids 100 residues or less in length), recombinant vectors, including, for example, plasm ids, cosmids, phage, viruses, and the like. Polynucleotides include, in certain aspects, regulatory sequences, isolated substantially away from their naturally occurring genes or protein encoding sequences. Polynucleotides may be RNA, DNA, analogs thereof, or a combination thereof.

In this respect, the term “gene,” “polynucleotide” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide of the following lengths: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs. It also is contemplated that a particular polypeptide from a given species may be encoded by nucleic acids containing natural variations that having slightly different nucleic acid sequences but, nonetheless, encode the same or substantially similar protein.

In particular embodiments, the disclosure provides isolated nucleic acid segments and recombinant vectors incorporating nucleic acid sequences that encode a Hla or any variant or fragment thereof. Thus, an isolated nucleic acid segment or vector containing a nucleic acid segment may encode, for example, a Hla or Hla(H35L) protein that is immunogenic. The term “recombinant” may be used in conjunction with a polypeptide or the name of a specific polypeptide, and this generally refers to a polypeptide produced from a nucleic acid molecule that has been manipulated in vitro or that is a replication product of such a molecule.

In other embodiments, the disclosure concerns isolated nucleic acid segments and recombinant vectors incorporating nucleic acid sequences that encode a Hla or Hla variant polypeptide or peptide that can be used to generate an immune response in a subject. In various embodiments the nucleic acids of the disclosure may be used in genetic vaccines.

The nucleic acid segments used in the present disclosure, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

The nucleic acid used in the present disclosure encodes Hla or any Hla variant or fragment. Such sequences may arise as a consequence of codon redundancy and functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by human may be introduced through the application of site-directed mutagenesis techniques, e.g., to introduce improvements to the antigenicity of the protein.

In certain other embodiments, the disclosure provides isolated nucleic acid segments and recombinant vectors that include within their sequence a contiguous nucleic acid sequence from SEQ ID NO:5. In some embodiments, the present disclosure provides a nucleic acid encoding any of the Hla peptides as disclosed herein.

Suitable methods for nucleic acid delivery to effect expression of compositions of the present disclosure are believed to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); or by desiccation/inhibition mediated DNA uptake (Potrykus et al., 1985). Through the application of techniques such as these, organelle(s), cell(s), tissue(s) or organism(s) may be stably or transiently transformed.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in an amino acid sequence, and in its underlying DNA coding sequence, and nevertheless produce a protein with like properties. It is thus contemplated by the inventors that various changes may be made in the DNA sequences of genes or nucleic acids without appreciable loss of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In certain embodiments, a Hla of the disclosure comprises the sequence set forth in SEQ ID NO:1, 2, 3, 4, 5, 6, 7, 8, 9, 10 or 11. In other embodiments, a Hla of the disclosure may have about 80% identity to SEQ ID NO: 1, 2, 3, 4, 5, 6, 7, 8, 9, 10 or 11. For example, a Hla of the disclosure may have about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity to SEQ ID NO: 1, 2, 3, 4, 5, 6, 7, 8, 9, 10 or 11. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the disclosure. Equally, BLAST protein searches may be performed with the BLASTX program to obtain amino acid sequences that are homologous to a polypeptide of the disclosure. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed.

In many instances, a biologically active variant will contain one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present disclosure and still obtain a functional molecule that encodes a variant or derivative polypeptide with desirable characteristics. When it is desired to alter the amino acid sequence of a polypeptide to create an equivalent, or even an improved, variant or portion of a polypeptide described herein, one skilled in the art will typically change one or more of the codons of the encoding DNA sequence.

The nucleic acid sequences which encode a Hla of the disclosure can be operatively linked to expression control sequences. “Operatively linked” refers to a juxtaposition wherein the components so described are in a relationship permitting them to function in their intended manner. An expression control sequence operatively linked to a coding sequence is achieved under conditions compatible with the expression control sequences. As used herein, the expression control sequences refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns, and maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

In one aspect, the present disclosure provides for a vector comprising a nucleic acid sequence encoding a Hla of the disclosure. In one aspect, the present disclosure is predicated, at least in part, on the ability of adeno-associated virus (AAV) vectors to be safely administered to humans and to provide persistent expression of a therapeutic transgene. The disclosure provides an adeno-associated virus (AAV) vector which comprises, consists essentially of, or consists of a nucleic acid sequence encoding a Hla polypeptide. When the AAV vector consists essentially of a nucleic acid sequence encoding a Hla polypeptide, additional components can be included that do not materially affect the AAV vector (e.g., genetic elements such as poly(A) sequences or restriction enzyme sites that facilitate manipulation of the vector in vitro). When the AAV vector consists of a nucleic acid sequence encoding a Hla polypeptide, the AAV vector does not comprise any additional components (i.e., components that are not endogenous to AAV and are not required to effect expression of the nucleic acid sequence to thereby provide the Hla).

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter. In a particular embodiment, the AAV contains a pair of inverted terminal repeats (ITRs) which flank at least one cassette containing a promoter which directs cell-specific expression (e.g. hepatocytes) operably linked to a heterologous gene. Heterologous in this context refers to any nucleotide sequence or gene which is not native to the AAV or B19 parvovirus (e.g. Hla). Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector. In some embodiments, the promoter directs cell-specific expression in the liver. Non-limiting examples include the al-antitrypsin (AT) promoter, thyroxine binding globulin promoter, human albumin promoter, liver-specific (LSP) promoter consisting of the 475 bp thyroid hormone binding globulin promoter and 2 copies of the 96 bp bikunin/α1-microglobulin enhancer, the DC190 promoter (728 bp) containing a 520 bp human albumin promoter and 2 copies of the 99 bp prothrombin enhancer or the DC172 promoter (1.272 kb) consisting of a 890 bp human (α1-antitrypsin promoter and 2 copies of the 160 bp a α1-microglobulin enhancer. In an exemplary embodiment, the cell-specific promoter is a liver-specific thyroxine binding globulin (TBG) promoter.

As used herein, the term “AAV vector” means a vector derived from an adeno-associated virus serotype. In non-limitation examples AAV vectors include, AAV1, AAV2, AAV3, AAV4, AAV5, AAV6, AAV7, AAV8, AAV9, and mutated forms thereof. AAV vectors can have one or more of the AAV wild-type genes deleted in whole or part, preferably the rep and/or cap genes, but retain functional flanking ITR sequences. Despite the high degree of homology, the different serotypes have tropisms for different tissues. In an exemplary embodiment, the AAV vector is AAV9.

An AAV vector, as disclosed herein, can be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316-327 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-6 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, B. J., Hum. Gene Ther., 16: 541-550 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71: 6823-33 (1997); Srivastava et al., J. Virol., 45: 555-64 (1983); Chiorini et al., J. Virol., 73: 1309-1319 (1999); Rutledge et al., J. Virol., 72: 309-319 (1998); and Wu et al., J. Virol., 74: 8635-47 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 73(2): 939-947 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (i.e., functionally substitute) corresponding sequences from other serotypes during production of AAV particles in mammalian cells.

Generally, the cap proteins, which determine the cellular tropicity of the AAV particle, and related cap protein-encoding sequences, are significantly less conserved than Rep genes across different AAV serotypes. In view of the ability Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1): 1-2 (2006); Gao et al., J. Virol., 78: 6381-6388 (2004); Gao et al., Proc. Natl. Acad. Sci. USA, 99: 11854-11859 (2002); De et al., Mol. Ther., 13: 67-76 (2006); and Gao et al., Mol. Ther., 13: 77-87 (2006).

In one embodiment, the AAV vector is generated using an AAV that infects humans (e.g., AAV2). Alternatively, the AAV vector is generated using an AAV that infects non-human primates, such as, for example, the great apes (e.g., chimpanzees), Old World monkeys (e.g., macaques), and New World monkeys (e.g., marmosets). Preferably, the AAV vector is generated using an AAV that infects a non-human primate pseudotyped with an AAV that infects humans. Examples of such pseudotyped AAV vectors are disclosed in, e.g., Cearley et al., Molecular Therapy, 13: 528-537 (2006). In one embodiment, an AAV vector can be generated which comprises a capsid protein from an AAV that infects rhesus macaques pseudotyped with AAV2 inverted terminal repeats (ITRs). In a particularly preferred embodiment, the inventive AAV vector comprises a capsid protein from AAV10 (also referred to as “AAVrh.10”), which infects rhesus macaques pseudotyped with AAV2 ITRs (see, e.g., Watanabe et al., Gene Ther., 17(8): 1042-1051 (2010); and Mao et al., Hum. Gene Therapy, 22: 1525-1535 (2011)).

An AAV vector, as disclosed herein, comprises a nucleic acid sequence encoding a Hla polypeptide. “Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

In some embodiments, a vector comprising a nucleic acid sequence encoding a Hla can be a plasmid, cosmid, yeast artificial chromosome (YAC), bacterial artificial chromosome (BAC), viral vector or bacteriophage. The vectors can provide for replication of Hla nucleic acids, expression of Hla polypeptides or integration of Hla nucleic acids into the chromosome of a host cell. The choice of vector is dependent on the desired purpose. Certain cloning vectors are useful for cloning, mutation and manipulation of the Hla nucleic acid. Other vectors are useful for expression of the Hla polypeptide, being able to express the polypeptide in large amounts for purification purposes or to express the Hla polypeptide in a temporal or tissue specific manner. The vector can also be chosen on the basis of the host cell, e.g., to facilitate expression in bacteria, mammalian cells, insect cells, fish cell (e.g., zebrafish) and/or amphibian cells. The choice of matching vector to host cell is apparent to one of skill in the art, and the types of host cells are discussed below. Many vectors or vector systems are available commercially, for example, the pET bacterial expression system (Invitrogen™, Carlsbad Calif.).

The vectors disclosed herein can be viral or non-viral vectors. For example, as discussed above the disclosed vectors can be viral vectors. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasm ids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain neurodegenerative diseases or disorders and cell populations by using the targeting characteristics of the carrier.

Vectors can include various components including, but not limited to, an origin of replication, one or more marker or selectable genes (e.g. GFP, neo), promoters, enhancers, terminators, poly-adenylation sequences, repressors or activators. Such elements are provided in the vector so as to be operably linked to the coding region of the Hla-encoding nucleic acid, thereby facilitating expression in a host cell of interest. Cloning and expression vectors can contain an origin of replication which allows the vector to replicate in the host cells. Vectors can also include a selectable marker, e.g., to confer a resistance to a drug or compliment complement deficiencies in growth. Examples of drug resistance markers include, but are not limited to, ampicillin, tetracycline, neomycin or methotrexate. Examples of other marker genes can be the fluorescent polypeptides such as one of the members of the fluorescent family of proteins, for example, GFP, YFP, BFP, RFP etc. These markers can be contained on the same vector as the gene of interest or can be on separate vectors and co-transfected with the vector containing the gene of interest.

The vector can contain a promoter that is suitable for expression of the Hla in mammalian cells, which promoter can be operably linked to provide for inducible or constitutive expression of a Hla polypeptide. Exemplary inducible promoters include, for example, the metallothionine promoter or an ecdysone-responsive promoter. Exemplary constitutive promoters include, for example, the viral promoters from cytomegalovirus (CMV), Rous Sarcoma virus (RSV), Simian virus 40 (SV40), avian sarcoma virus, the beta-actin promoter and the heat-shock promoters. The promoter can be chosen for its tissue specificity. Certain promoters only express in certain tissues, and when it is desirable to express the polypeptide of interest only in a selected tissue, one of these promoters can be used. The choice of promoter will be apparent to one of skill in the art for the desired host cell system.

The vector encoding a Hla can be a viral vector. Examples of viral vectors include retroviral vectors, such as: adenovirus, simian virus 40 (SV40), cytomegalovirus (CMV), Moloney murine leukemia virus (MoMuLv), Rous Sarcoma Virus (RSV), lentivirus, herpesvirus, poxvirus and vaccinia virus. A viral vector can be used to facilitate expression in a target cell, e.g., for production of Hla or for use in therapy (e.g., to deliver a Hla to a subject by expression from the vector). Where used for therapy, Hla-encoding vectors (e.g, viral vectors), can be administered directly to the patient via an appropriate route or can be administered using an ex vivo strategy using subject cells (autologous) or allogeneic cells, which are suitable for administration to the patient to be treated.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. The viral vectors may be formulated in pharmaceutical compositions as those described above

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Feigner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

Host cells modified to provide for expression of a Hla peptide disclosed herein are also contemplated. Such host cells can be modified to express a Hla polypeptide from either an episomal or genomically integrated nucleic acid. Such host cells can be produced by any suitable method, e.g., electroporation, transfection or transformation with a vector encoding a Hla polypeptide. Host cells can be selected according to a desired use (e.g., mammalian cell expression), and modified to provide for Hla expression according to methods well known in the art. Techniques for introducing the vectors into host cells and subsequent culture of the host cells are well known in the art.

Host cells (e.g., mammalian host cells) suitable for replication and expression of Hla containing vectors are provided, wherein the cells may be stably or transiently transfected and/or stably or transiently express a Hla. Such Hla-expressing mammalian cells find use in, for example, production of a Hla. Production of Hla in mammalian cells can provide for post-translational modifications of the Hla and/or to heterologous amino acids to which it may be fused (e.g., glycosylation, cleavage of signal peptide (if present)). In addition, mammalian cell lines can be selected for use in replicating, packaging and producing high titers of virus particles which contain a Hla of interest or nucleic acid-encoding a Hla. Such Hla containing viruses can then be used to provide for delivery of Hla-encoding nucleic acids and Hla polypeptides to a subject in need thereof.

Exemplary host cells include bacteria, yeast, mammalian cells (e.g., human cells or cell lines), insect cells, and the like. Examples of bacterial host cells include E. coli and other bacteria which can find use in cloning, manipulation and production of Hla nucleic acids or the production of Hla polypeptide. Examples of mammalian cells include, but are not limited to, Chinese hamster ovary (CHO) cells, HEK 293 cells, human cervical carcinoma cells (Hela), canine kidney cells (MDCK), human liver cells (HepG2), baby hamster kidney cells (BHK), and monkey kidney cells (CV1).

b) Components of the Composition

The present disclosure also provides pharmaceutical compositions. The pharmaceutical composition comprises a Hla as disclosed herein, as an active agent, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In each of the embodiments described herein, a composition of the disclosure may optionally comprise one or more additional drug or therapeutically active agent in addition to the Hla. Thus, in addition to the therapies described herein, one may also provide to the subject other therapies known to be efficacious for treatment of the disease, disorder, or condition.

(i) Diluent

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

(ii) Binder

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

(iii) Filler

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

(iv) Buffering Agent

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

(v) pH Modifier

In various embodiments, the excipient may be a pH modifier. By way of non-limiting example, the pH modifying agent may be sodium carbonate, sodium bicarbonate, sodium citrate, citric acid, or phosphoric acid.

(vi) Disintegrant

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

(vii) Dispersant

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

(viii) Excipient

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

(ix) Lubricant

In a further embodiment, the excipient may be a lubricant. Non-limiting examples of suitable lubricants include minerals such as talc or silica; and fats such as vegetable stearin, magnesium stearate, or stearic acid.

(x) Taste-Masking Agent

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

(xi) Flavoring Agent

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

(xii) Coloring Agent

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

(d) Administration

(i) Dosage Forms

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18th ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including cutaneous, subcutaneous, intraocular, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules, and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols, or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles, and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising the Hla or variant thereof, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present disclosure. Non-limiting examples of suitable structured fluid delivery systems may include nanoparticles, liposomes, microemulsions, micelles, dendrimers, and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the Hla, in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the composition comprising the Hla or variant thereof may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3, 3, 3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the one or more of a tricyclic antipsychotic, vasodilator, antibiotic/antiseptic, aryl piperazine or derivatives thereof, may be prepared by any known method of preparing liposomes for drug delivery, such as, for example, detailed in U.S. Pat. Nos. 4,241,046; 4,394,448; 4,529,561; 4,755,388; 4,828,837; 4,925,661; 4,954,345; 4,957,735; 5,043,164; 5,064,655; 5,077,211; and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of one or more of a proteotoxicity reducing agent or derivatives thereof, concentration and composition of lipid, concentration of multivalent cations, rate of mixing, presence of and concentration of solvent.

In another embodiment, a composition of the disclosure may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the disclosure generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The one or more of a tricyclic antipsychotic, vasodilator, antibiotic/antiseptic, aryl piperazine or derivatives thereof may be encapsulated in a microemulsion by any method generally known in the art.

In yet another embodiment, the Hla, may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the disclosure therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the disclosure. Generally, the size of dendrimers may range from about 1 nm to about 100 nm.

Generally, a safe and effective amount of a Hla is, for example, that amount that would cause the desired effect in a subject while minimizing undesired side effects. In various embodiments, an effective amount of Hla described herein can substantially induce an immune response in a subject.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The present disclosure encompasses pharmaceutical compositions comprising compounds as disclosed above, so as to facilitate administration and promote stability of the active agent. For example, a compound of this disclosure may be admixed with at least one pharmaceutically acceptable carrier or excipient resulting in a pharmaceutical composition which is capably and effectively administered (given) to a living subject, such as to a suitable subject (i.e. “a subject in need of treatment” or “a subject in need thereof”). For the purposes of the aspects and embodiments of the disclosure, the subject may be a human or any other animal.

In some embodiments, there is between about 0.001 mg and about 10 mg of total protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 μg/ml, mg/ml, or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% may be Hla protein.

The present disclosure contemplates the administration of a Hla polypeptide or peptide to affect a preventative therapy against the development of a disease or condition associated with infection by a staphylococcus pathogen.

The present disclosure describes polypeptides, peptides, and proteins for use in various embodiments of the present disclosure. For example, specific polypeptides are assayed for their abilities to elicit an immune response. In specific embodiments, all or part of the proteins of the disclosure can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence which encodes a peptide of the disclosure is inserted into an expression vector, transformed or transfected into an appropriate host cell and cultivated under conditions suitable for expression.

One embodiment of the disclosure includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide described herein may be employed. The generation of recombinant expression vectors, and the elements included therein, are discussed herein. Alternatively, the protein to be produced may be an endogenous protein normally synthesized by the cell used for protein production.

Another embodiment of the present disclosure uses autologous B lymphocyte cell lines, which are transfected with a viral vector that expresses an immunogen product, and more specifically, a protein having immunogenic activity. Other examples of mammalian host cell lines include, but are not limited to Vero and HeLa cells, other B- and T-cell lines, such as CEM, 721.221, H9, Jurkat, Raji, as well as cell lines of Chinese hamster ovary, W138, BHK, COS-7, 293, HepG2, 3T3, RIN and MDCK cells. In addition, a host cell strain may be chosen that modulates the expression of the inserted sequences, or that modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed.

A number of selection systems may be used including, but not limited to HSV thymidine kinase, hypoxanthine-guanine phosphoribosyltransferase, and adenine phosphoribosyltransferase genes, in tk-, hgprt- or aprt-cells, respectively. Also, anti-metabolite resistance can be used as the basis of selection: dhfr, which confers resistance to trimethoprim and methotrexate; gpt, which confers resistance to mycophenolic acid; neo, which confers resistance to the aminoglycoside G418; and hygro, which confers resistance to hygromycin.

Animal cells can be propagated in vitro in two modes: as non-anchorage-dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent cells.

The present disclosure includes methods for preventing or ameliorating staphylococcus infections. Embodiments of the disclosure include preventing or ameliorating staphylococcal pneumonia. As such, the disclosure contemplates vaccines for use in both active and passive immunization embodiments. Immunogenic compositions, proposed to be suitable for use as a vaccine, may be prepared most readily directly from immunogenic Hla peptide or protein prepared in a manner disclosed herein. Preferably the antigenic material is extensively dialyzed to remove undesired small molecular weight molecules and/or lyophilized for more ready formulation into a desired vehicle. The disclosure includes compositions that can be used to induce an immune response against a polypeptide or peptide derived from a Hla peptide or protein so as to protect against infection by a staphylococcus and against developing a condition or disease caused by such. In certain aspects a composition is formulated to be administered to a mucosal surface, e.g., an aerosol formulation.

Alternatively, other viable and important options for a protein/peptide-based vaccine involve introducing nucleic acids encoding the antigen(s) as DNA vaccines. In this regard, recent reports described construction of recombinant vaccinia viruses expressing either 10 contiguous minimal CTL epitopes (Thomson, 1996) or a combination of B cell, CTL, and TH epitopes from several microbes (An, 1997), and successful use of such constructs to immunize mice for priming protective immune responses. Thus, there is ample evidence in the literature for successful utilization of peptides, peptide-pulsed APCs, and peptide-encoding constructs for efficient in vivo priming of protective immune responses. The use of nucleic acid sequences as vaccines is exemplified in U.S. Pat. Nos. 5,958,895 and 5,620,896.

The preparation of vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such vaccines are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, vaccines are formulated with a combination of substances, as described in U.S. Pat. Nos. 6,793,923 and 6,733,754, which are incorporated herein by reference.

Vaccines may be conventionally administered parenterally, mucosally, intranasally, by inhalation, and/or by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

The polypeptides and polypeptide-encoding DNA constructs may be formulated into a vaccine as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, vaccines are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, mucosally, intranasally, by inhalation, by injection and the like. The dosage of the vaccine will depend on the route of administration and will vary according to the size and health of the subject.

A given composition may vary in its immunogenicity. It is often necessary therefore to boost the host immune system, as may be achieved by coupling a peptide or polypeptide to a carrier. Exemplary and preferred carriers are keyhole limpet hemocyanin (KLH) and bovine serum albumin (BSA). Other albumins such as ovalbumin, mouse serum albumin, or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde, and bis-biazotized benzidine.

The immunogenicity of polypeptide or peptide compositions can be enhanced by the use of non-specific stimulators of the immune response, known as adjuvants. Suitable adjuvants include all acceptable immunostimulatory compounds, such as cytokines, toxins, or synthetic compositions.

A number of adjuvants can be used to enhance an antibody response against a Hla peptide or protein. Adjuvants can (1) trap the antigen in the body to cause a slow release; (2) attract cells involved in the immune response to the site of administration; (3) induce proliferation or activation of immune system cells; or (4) improve the spread of the antigen throughout the subject's body.

Adjuvants include, but are not limited to, oil-in-water emulsions, water-in-oil emulsions, mineral salts, polynucleotides, and natural substances. Specific adjuvants that may be used include IL-1, IL-2, IL-4, IL-7, IL-12, y-interferon, GMCSP, BCG, aluminum hydroxide or other aluminum compound, MDP compounds, such as thur-MDP and nor-MDP, CGP (MTP-PE), lipid A, and monophosphoryl lipid A (MPL). Other adjuvants that may be used include RIBI, which contains three components extracted from bacteria, MPL, trehalose dimycolate (TDM), and cell wall skeleton (CWS) in a 2% squalene/Tween 80 emulsion. MHC antigens may even be used. Others adjuvants or methods are exemplified in U.S. Pat. Nos. 6,814,971, 5,084,269, 6,656,462, each of which is incorporated herein by reference).

Various methods of achieving adjuvant affect for the vaccine includes use of agents such as aluminum hydroxide or phosphate (alum), commonly used as about 0.05 to about 0.1% solution in phosphate buffered saline, admixture with synthetic polymers of sugars (Carbopol®) used as an about 0.25% solution, aggregation of the protein in the vaccine by heat treatment with temperatures ranging between about 70° to about 101° C. for a 30-second to 2-minute period, respectively. Aggregation by reactivating with pepsin-treated (Fab) antibodies to albumin; mixture with bacterial cells (e.g., C. parvum), endotoxins or lipopolysaccharide components of Gram-negative bacteria; emulsion in physiologically acceptable oil vehicles (e.g., mannide mono-oleate (Aracel A)); or emulsion with a 20% solution of a perfluorocarbon (Fluosol-DA®) used as a block substitute may also be employed to produce an adjuvant effect.

Exemplary, often preferred adjuvants include complete Freund's adjuvant (a non-specific stimulator of the immune response containing killed Mycobacterium tuberculosis), incomplete Freund's adjuvants, and aluminum hydroxide.

In addition to adjuvants, it may be desirable to co-administer biologic response modifiers (BRM) to enhance immune responses. BRMs have been shown to upregulate T cell immunity or downregulate suppresser cell activity. Such BRMs include, but are not limited to, Cimetidine (CIM; 1200 mg/d) (Smith/Kline, PA); low-dose Cyclophosphamide (CYP; 300 mg/m2) (Johnson/Mead, NJ) and cytokines such as y-interferon, IL-2, or IL-12 or genes encoding proteins involved in immune

In certain embodiments, the present disclosure concerns compositions comprising one or more lipids associated with a nucleic acid or a polypeptide/peptide. A lipid is a substance that is insoluble in water and extractable with an organic solvent. Compounds other than those specifically described herein are understood by one of skill in the art as lipids, and are encompassed by the compositions and methods of the present disclosure. A lipid component and a non-lipid may be attached to one another, either covalently or non-covalently.

A nucleic acid molecule or a polypeptide/peptide, associated with a lipid may be dispersed in a solution containing a lipid, dissolved with a lipid, emulsified with a lipid, mixed with a lipid, combined with a lipid, covalently bonded to a lipid, contained as a suspension in a lipid or otherwise associated with a lipid. A lipid or lipid-Hla-associated composition of the present disclosure is not limited to any particular structure. For example, they may also simply be interspersed in a solution, possibly forming aggregates which are not uniform in either size or shape. In another example, they may be present in a bilayer structure, as micelles, or with a “collapsed” structure. In another non-limiting example, a lipofectamine (Gibco BRL)-poxvirus or Superfect (Qiagen)-poxvirus complex is also contemplated.

In certain embodiments, a composition may comprise about 1%, about 2%, about 3%, about 4% about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or any range there between, of a particular lipid, lipid type, or non-lipid component such as an adjuvant, antigen, peptide, polypeptide, sugar, nucleic acid or other material disclosed herein or as would be known to one of skill in the art. In a non-limiting example, a composition may comprise about 10% to about 20% neutral lipids, and about 33% to about 34% of a cerebroside, and about 1% cholesterol. In another non-limiting example, a liposome may comprise about 4% to about 12% terpenes, wherein about 1% of the micelle is specifically lycopene, leaving about 3% to about 11% of the liposome as comprising other terpenes; and about 10% to about 35% phosphatidyl choline, and about 1% of a non-lipid component. Thus, it is contemplated that compositions of the present disclosure may comprise any of the lipids, lipid types or other components in any combination or percentage range.

The compositions and related methods of the present disclosure, particularly administration of a Hla to a patient/subject, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline, oxacillin, vancomycin or various combinations of antibiotics. In addition, administration of a Hla protein or anti-Hla antibodies to a patient/subject may be used in combination with the administration of antivirulence agents, such as RIP.

In one aspect, it is contemplated that a Hla composition is used in conjunction with antibacterial and/or antivirulence treatment. Alternatively, the therapy may precede or follow the other agent treatment by intervals ranging from minutes to weeks. In embodiments where the other agents and/or a proteins or polynucleotides are administered separately, one would generally ensure that a significant period of time did not expire between each delivery, such that the agent and the composition of the present disclosure would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6 or 7) to several weeks (1, 2, 3, 4, 5, 6, 7 or 8) lapse between the respective administrations.

Various combinations may be employed, for example antibiotic therapy is “A” and the immunogenic molecule or antibody given as part of an immune or passive immune therapy regime, respectively, such as a Hla antigen, is “B”: A/B/A B/A/B B/B/A A/A/B A/B/B B/A/A A/B/B/B B/A/B/B B/B/B/A B/B/NB A/A/B/B A/B/NB A/B/B/A B/B/A/A B/A/B/A B/A/A/B A/A/A/B B/A/A/A A/B/A/A A/A/B/A.

In some embodiments, pharmaceutical compositions are administered to a subject. Different aspects of the present disclosure involve administering an effective amount of a composition to a subject. In some embodiments of the present disclosure, a Hla polypeptide or peptide may be administered to the patient to protect against infection by one or more staphylococcus pathogens. Alternatively, a nucleic acid sequence or expression vector comprising the same which encode one or more such polypeptides or peptides may be given to a subject as a preventative treatment. Additionally, such compounds can be administered in combination with an antibiotic and/or antivirulence agent. Such compositions will generally be dissolved or dispersed in a pharmaceutically acceptable carrier or aqueous medium.

The active compounds of the present disclosure can be formulated for parenteral administration, e.g., formulated for injection via the intravenous, intramuscular, sub-cutaneous, or even intraperitoneal routes. The preparation of an aqueous composition that contains a composition or compositions of the present disclosure will be known to those of skill in the art in light of the present disclosure. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Additional formulations of pharmaceutical delivery systems may be in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Remington's Pharmaceutical Sciences, Mack Publishing Co., Easton Pa., 16 Ed ISBN: 0-912734-04-3, latest edition, incorporated herein by reference in its entirety, provides a compendium of formulation techniques as are generally known to practitioners. A suitable pharmaceutically acceptable carrier to maintain optimum stability, shelf-life, efficacy, and function of the delivery system would be apparent to one of ordinary skill in the art.

Controlled-release (or sustained-release) preparations may be formulated to extend the activity of the agent(s) reduce the dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### II. Methods

The present disclosure provides the importance of mediating immunity to a bacterial pathogen prior to a first infection by the pathogen. As described herein, the effects of bacterial antigens on T cell-mediated immunity occur during initial exposure, and therefore the T cell repertoire can be perturbed by colonization or infection in infancy. As a result, individuals with bacterial pathogen exposure harbor a preexisting T cell repertoire influenced by the pathogen. Thus, a post-exposure vaccine may not be capable of favorably altering the diversity of the T cell response or specific effector functions necessary for protective immunity. Accordingly, the present disclosure encompasses, in general, of vaccinating subjects to a bacterial antigen prior to the subjects first exposure to the bacterial pathogen, for example, methods such as maternal immunization and/or infant vaccination to generate population-level protective immunity without the defect seen in adaptive immunity generated by a first infection.

By removing the suppressive effects of the bacterial antigen (e.g., Hla) on host immune function, immunization against Hla may expand antigen-specific T cell diversity and allow natural bacterial pathogen exposure to amplify the T cell repertoire rather than elicit tolerogenic or suppressive responses. Thus, the present disclosure provides methods of generating an immune response in a subject by administering to the subject a composition comprising a bacterial antigen prior to the first infection of the subject by the pathogen. Accordingly, the present disclosure provides methods to reduce or prevent tolerogenic or suppressive responses T-cell responses of a subject to a bacterial pathogen.

As described herein, the methods generally comprise active immunization of newborns at the time of birth followed by booster immunizations during the primary series in infancy and early childhood. For example, the methods as disclosed herein include methods for reducing or preventing a S. aureus infection in a subject, the methods generally comprising administering to the subject compositions as described herein at birth or shortly thereafter. In some embodiments, at birth or shortly after birth may include but is not limited to, within about 1, 2, 3, 4, 5, 10, 15, 20, 25, 30, 35, 40, 45, 50, 55 minutes after birth. In some embodiments, shortly after birth may include but is not limited to, within about 1, 2, 3, 4, 5, 6, 7, 8, 9, 10, 11, 12, 13, 14, 15, 16, 17, 18, 19, 20, 21, 22, 23, 24 hours after birth. In some embodiments, shortly after birth includes within about 1, 2, 3, 4, 5, 6, 7 days after birth. In some embodiments, shortly after birth includes within about 1, 2, 3, 4, 5, 6, 7, or 8 weeks after birth. The methods further comprise administering to the subject a composition as disclosed herein one or more times following the first administration at birth or shortly after birth. In some embodiments, the one or more additional administrations include within about 1, 2, 3, 4, 5, 6, 7 days after the first administration. In some embodiments, the one or more additional administrations include within about 1, 2, 3, 4, 5, 6, 7, 8, 9, 10, 11, or 12 months after the first administration. In some embodiments, the one or more additional administrations include within about 1, 2, 3, 4, 5, 6, 7, 8, 9, 10, 11, or 12 years after the first administration.

In another embodiment, the methods provide maternal immunization for neonatal protection, coupled with immunization during the primary series in infancy and early childhood. For example, the methods as disclosed herein include methods for reducing or preventing a S. aureus infection in a subject, the methods generally comprising administering to the mother of a subject a composition as disclosed herein while the subject is in utero. In utero is a Latin term literally meaning “in the womb” or “in the uterus”. In some embodiments, the methods elicit transplacental transfer of anti-Hla neutralizing antibodies. In some embodiments, the mother is administered the compositions while in the second or third trimester of pregnancy. Thus, a mother can be administered the composition within about 13, 14, 15, 16, 17, 18, 19, 20, 21, 22, 23, 24, 25, 26, 27, 28, 29, 30, 31, 32, 33, 34, 35, 36, 37, 38, 39, or 40 weeks of pregnancy. The methods further comprise administering to the subject at birth or shortly after birth a composition as disclosed herein one or more times following the first administration to the mother while the subject was in utero.

As discussed above, the disclosure concerns evoking an immune response in a subject against a Hla or a variant or fragment thereof. In one embodiment, the immune response can protect against or treat a subject having, suspected of having, or at risk of developing an infection or related disease. In some embodiments, the method includes evoking an immune response in a subject prior to the subject's first exposure to S. aureus. In non-limiting examples, the immune response may be evoked in the subject in utero and/or at birth or shortly thereafter.

In some embodiments of the disclosure, compositions as disclosed herein generate an immune response in the subject thereby conferring protective immunity on a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” refers to the development of a humoral (antibody mediated), cellular (mediated by antigen-specific T cells or their secretion products) or both humoral and cellular response directed against a protein, peptide, or polypeptide of the disclosure in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

A method of the present disclosure includes treatment for a disease or condition caused by a staphylococcus pathogen, as well as prevention of or reduction in infection so as to prevent or minimize the extent of exposure to the pathogen. An immunogenic polypeptide of the disclosure can be given to induce an immune response in a person prior to the subject's first exposure to staphylococcus (e.g., while the subject is in utero).

In some embodiments, the treatment is administered in the presence of adjuvants or carriers in the absence or substantial absence of other staphylococcal antigens and/or proteins. Furthermore, in some examples, treatment comprises administration of other agents commonly used against bacterial infection, such as one or more antibiotics.

Administration of a Hla or variant thereof can occur as a single event or over a time course of treatment. For example, one or more of a Hla can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more. Compositions may be administered 1, 2, 3, 4, 5, 6, 7, 8, 9, 10, 11, 12, 13, 14, 15, 16, 17, 18, 19, 20 or more times, and/or they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months, or any range or combination derivable therein.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

As various changes could be made in the above-described materials and methods without departing from the scope of the disclosure, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Staphylococcus aureus Toxin Suppresses Antigen-Specific T Cell Responses

Staphylococcus aureus remains a leading cause of human infection. These infections frequently recur when the skin is a primary site of infection, especially in infants and children. In contrast, invasive staphylococcal disease is less commonly associated with reinfection, suggesting that tissue-specific mechanisms govern the development of immunity. Knowledge of how S. aureus manipulates protective immunity has been hampered by a lack of antigen-specific models to interrogate the T cell response. Using a chicken egg OVA-expressing S. aureus strain to analyze OVA-specific T cell responses, the present Example demonstrates that primary skin infection is associated with impaired development of T cell memory. Conversely, invasive infection induced antigen-specific memory and protected against reinfection. This defect in adaptive immunity following skin infection was associated with a loss of dendritic cells (DCs), attributable to S. aureus α-toxin (Hla) expression. Gene- and immunization-based approaches to protect against Hla during skin infection restored the T cell response. Within the human population, exposure to α-toxin through skin infection may modulate the establishment of T cell-mediated immunity, adversely affecting long-term protection. This Example provides support that vaccination targeting S. aureus is most effective if delivered prior to initial contact with the organism.

### Methods

Mice. All mice were housed in specific pathogen-free animal facilities. C57BL/6J mice were purchased from Jackson Laboratories. μMT mice were generously provided by Dr. Michael Diamond. OT-II CD45.1⁺ mice were kindly gifted by Dr. Anne Sperling. For all infections, age-matched 4-6-week old males and females were studied.

Bacterial strains and cultures. S. aureus USA300/LAC was engineered to express chicken egg ovalbumin (USA300_{OVA}) by cloning OVA₁₃₉₋₃₈₆ into the pKL plasmid. pKL was generated by modifying pww412 to include an improved translation initiation region with an optimized Shine-Dalgarno sequence and translation enhancer. OVA₁₃₉₋₃₈₆ was cloned from a S. aureus codon-optimized chicken egg ovalbumin sequence (Geneart) using 5′ primer TCGCATATGAAAACACGTATAGTCAGCTCAGTAACAA CAACACTATTGCTAGGTTCCATATTAATGAATCCTGTCGCTAATGCCGCAGATCAA GCACGTGAATTA (SEQ ID NO: 12) which includes the S. aureus Hla signal sequence, and 3′ primer TCTGGATCCTTATGGTGAAACACAACGACC (SEQ ID NO: 13). The resultant product was ligated into pKL to form pKLOVA. Strains of USA300 harboring pKL (USA300_{CTL}) or pKLOVA (USA300_{OVA}) as well as a Hla-deficient variant of USA300 (USA300_{OVA}hla::erm) were generated by electroporation utilizing standard methodologies. For infections, USA300 strains were grown overnight with rotation at 37° C. in tryptic soy broth (TSB, Sigma), subcultured and prepared as previously described. Media for growth of USA300hla::erm was supplemented with 40 μg/ml erythromycin, while media for strains harboring the pKL plasmid was supplemented with 20 μg/ml chloramphenicol. For all strains, OD₆₀₀ was measured to assess bacterial density, which was verified by serial plating on tryptic soy agar (TSA) plates to quantify colony forming units (CFU) per ml of culture.

Bacterial infections. For subcutaneous infections, mice were infected with 1×10⁸ CFU USA300 in 50 μl PBS. For intravenous infections, mice were infected with 5×10⁶ CFU USA300 in 100 μl PBS via retroorbital route. For evaluation of infection outcome with identical inocula, mice received 5×10⁶ CFU for both skin infection and intravenous infection. In experiments utilizing S. aureus strains harboring pKLCTL or pKLOVA, 0.5 mg/ml chloramphenicol and 1% sucrose solution drinking water was given to mice one day prior to infection, and maintained for 15 days following primary infection, or throughout the course of secondary infection. For experiments to assess memory T cell responses, water was switched back to normal drinking on day 14 of the experiment. On designated days post re-challenge, skin lesions were punch biopsied with an 8 mm punch biopsy (Integra Miltex) and placed in 1 ml of 0.1% Triton-X PBS solution then immediately processed using the Bio-Gen Pro200 Homogenizer (Pro Scientific). Homogenates were plated on TSA plates to quantify CFU values.

T cell adoptive transfer and T cell depletion. One day prior to infection, lymph nodes and spleens from OT-II CD45.1⁺ males were harvested for CD4⁺ T cell isolation. To extract OT-IIs, the CD4⁺ T cell Isolation Kit Miltenyi) was used according to manufacturer instructions. Approximately 1×10⁵ CD45.1⁺ OT-II cells were suspended in PBS and then transferred via retro-orbital injection into 5-week old C57BL/6J recipients (CD45.2⁺). The next day, recipients were infected as described. For CD4⁺ T cell depletion, 100 μg anti-CD4 monoclonal antibody (GK1.5, University of Chicago Fitch Monoclonal Antibody Facility) or an isotype-matched control antibody was delivered by intraperitoneal injection to each mouse beginning 6 days prior to intravenous infection every 3 days for 7 total doses.

OT-II cytokine assay. Lymph nodes and spleens from mice were harvested, and splenic RBCs were lysed using an ammonium chloride lysis solution. Cells were then Fc CD16/32 blocked on ice for 10 minutes and then incubated with CD45.1 biotinylated antibody (eBioscience) at 1:400 concentration on ice for 30 minutes. Samples were then washed in FACS buffer, resuspended in 300 μl FACS buffer, and 50 μl of streptavidin (Miltenyi) magnetic beads were added per sample for 30 minutes of incubation on ice. Gentle vortexing was performed for a few seconds every 10 minutes to ensure proper bead mixing. Cells were then washed and resuspended in FACS buffer and passed through LS MACS column for positive selection of CD45.1 OT-II cells. Positively selected cells were plated in 96 well tissue culture plates and incubated for 4 hours at 37° C. and 5% CO2 with PMA (50 ng/ml), ionomycin (500 ng/ml), Golgi stop and plug (Biolegend). Cells were then surface stained, fixed, permeabilized and stained per BD manufacturer protocol.

Skin histology. Skin lesions harvested by punch biopsy were extracted from mice after euthanasia, then fixed in 10% formalin (VWR, Avantor). Samples were embedded, sectioned, and stained with hematoxylin and eosin (Nationwide Histology).

Tissue Isolation and Flow Cytometric Staining. Skin lesions were punch biopsied, and blunt removal of fat tissue was performed with forceps. Tissue was minced and digested in RPMI (Gibco) containing 0.25 mg/ml Liberase TL (Roche Diagnostic Corp), 100 μM b-mercaptoethanol, 20 μM HEPES, 100 U/ml penicillin and 100 μg/ml streptomycin, and incubated for 2-3 hours at 37° C. and 5% CO2. For dendritic cell studies, draining axillary, brachial and subinguinal lymph nodes were extracted and digested with 0.1 mg/ml Liberase TL (Roche), 100 μM b-mercaptoethanol, 20 μM HEPES, 100 U/ml penicillin and 100 μg/ml streptomycin, and incubated for 30 minutes at 37° C. and 5% CO2. Tissue and lymph nodes were gently strained through a 40 μm cell strainer in single cell suspensions, and then incubated with anti-CD16/32/FC block (Biolegend) in fluorescence activated cell sorting buffer (FACS, 1% BSA, 0.1% NaN₃, 5 mM EDTA) for 15 minutes on ice. Cells were stained in FACS buffer on ice for 30-45 minutes and then analyzed on the cytometer, or fixed in 1% paraformaldehyde solution. For tissue, live-dead staining was performed before blockade of non-specific binding. Briefly, cells were incubated with Live Dead Fixable Aqua (Thermo Fisher) at 1:1000 in PBS for 30 minutes at 4° C. in the dark. Cells were washed twice in FACS buffer before incubation with FC block. Flow cytometric analysis was performed on a BD LSR Fortessa II. Murine cell suspensions were incubated with fluorochrome-conjugated antibodies (Biolegend) against the following surface markers: CD45 (30-F11), CD45.1 (A20), CD62L (MEL-14), CD4 (RM4-4, GK1.5), TCRb(H57-597), CD44 (IM7), CD8b (Ly-3), MHCII IA/I-E (M5/114.15.2), CD103 (2E7), CD207/Langerin (4C7), CD11c (N418), CD11 b (M1/70), anti-CD16/32 (2.4G2), IFNγ (XMG1.2), IL-17A (TC11-18H10.1), IL-10 (JES5-16E3), IL-4 (11B11). Cell counts were enumerated using AccuCount beads (Spherotech) according to manufacturer instructions.

Memory T cell isolation and quantification. OT-IIs were isolated by positive selection as detailed above. Due to the low numbers of OT-IIs isolated per mouse in long-term memory experiments, positively selected cells from 5 mice per group were pooled together, stained for extracellular markers specific to CD45.1 OT-II cells, and analyzed by flow cytometry. For each experimental group, the total number of OT-IIs collected was then divided by the number of mice to obtain the mean, total number of OT-IIs per mouse per group.

Immunization. For active immunization, 4-week old mice received 20 μg HlaH35L protein in complete Freund's adjuvant on day 0 via intramuscular route, followed by a boost with 20 μg HlaH35L protein in incomplete Freund's adjuvant on day 10 prior to infection on day 21. HlaH35L was prepared as previously described. Pre-immunization and day 20 sera were collected to assess antibody production. For maternal immunization studies, 8-week old female C57BL/6J mice were immunized on day 0 via intramuscular route with 20 μg of HlaH35L protein in complete Freund's adjuvant followed by a boost with 20 μg HlaH35L protein in incomplete Freund's adjuvant 14 days post-mating. Pups born to immunized dams were weaned at day 21 of life, then infected between 4-6 weeks of age. For all studies, adjuvant alone was delivered to control immunized mice.

Serum antibody quantification. Mice were bled on day 35 post primary infection by submandibular puncture using 5 mm Goldenrod Animal Lancets. Blood was kept on ice during collection and sera was extracted using (Sarstedt Micro tube 1.1 ml Z-Gel) serum collecting tubes and spun down per manufacturer protocol. Sera was plated as a dilution series in antigen-coated Costar Easy Wash ELISA 96 well plates. Coating was done overnight at 4° C. with either purified HlaH35L (1 μg/ml) or staphylococcal lysate (5 μg/ml) in PBS solution. To prepare staphylococcal lysate for coating, a staphylococcal Protein A-deficient S. aureus strain was cultivated overnight at 37° C. with shaking in Luria-Bertani (LB) medium. 1×1010 bacteria were harvested, washed with PBS, and resuspended in 200 of PBS buffer containing protease inhibitor cocktail tablets (Complete, Roche Diagnostics, Mannheim, Germany) and 20 μg of lysostaphin (Sigma-Aldrich, Germany). After enzymatic digestion at 37° C. for 30 min, the cells were disrupted by sonication using a microsonicator (Sonifier 250). Protein concentration was determined using DC protein assay kit (Bio-Rad) and lysate concentration was adjusted to 5 μg/ml in PBS prior to coating of ELISA plates. Blocking was performed with 0.1% BSA in PBS solution for 1 hour at room temperature or overnight at 4° C. After serum incubation, plates were washed 3 times with 0.05% Tween 20/PBS solution and patted dry prior to the addition of HRP-conjugated goat antimouse IgG in 0.1% BSA/PBS solution at 1:20,000 dilution for 45 minutes at room temperature. Plates were then washed 5 times with 0.05% Tween 20/PBS solution and developed with TMB substrate following manufacturer (ThermoFisher) recommended protocol. Absorbance (OD450) was measured using a microplate reader (Tecan Infinite M200Pro), and data analysis was performed using PRISM software to determine halfmaximal titers of each sample.

### Results

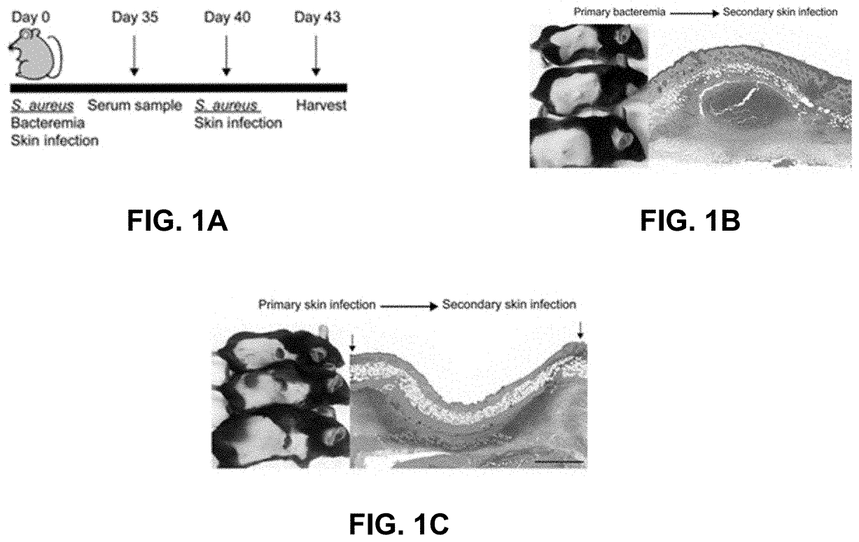

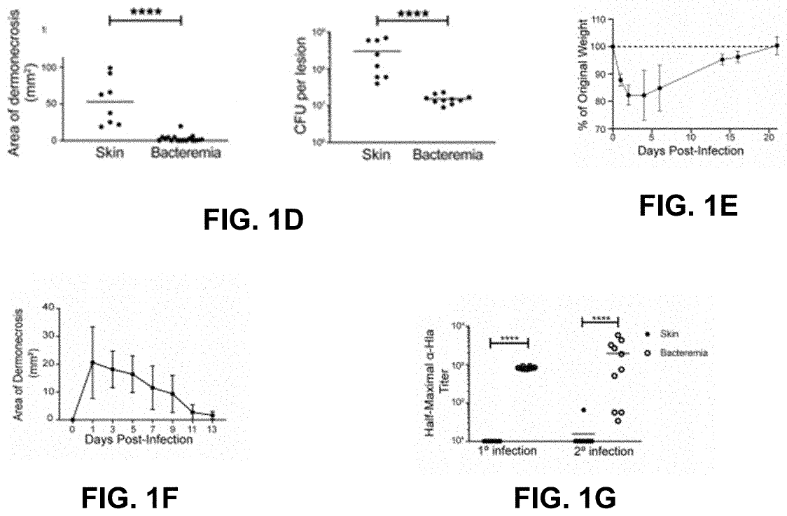

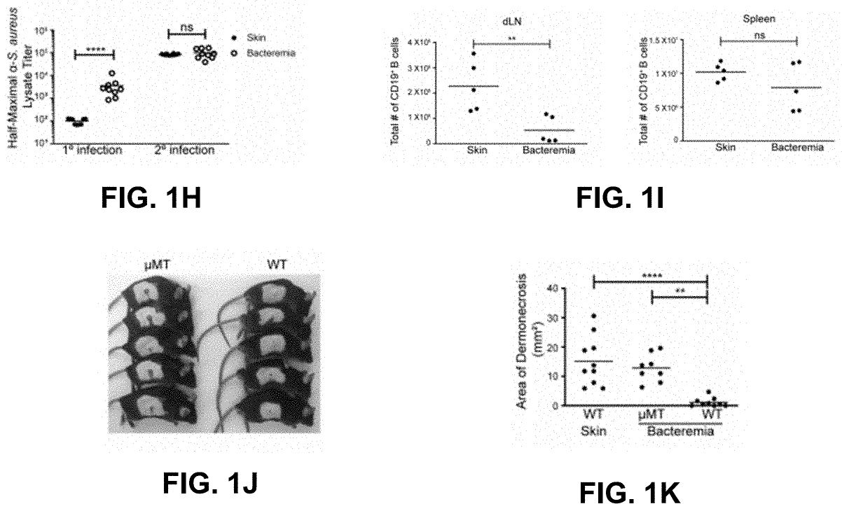

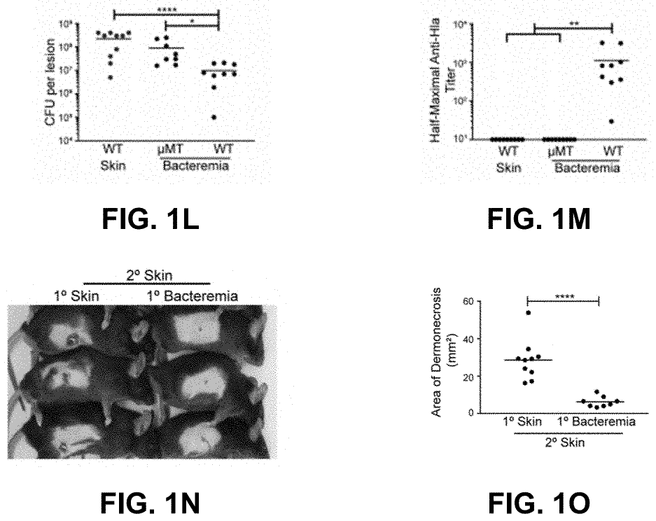

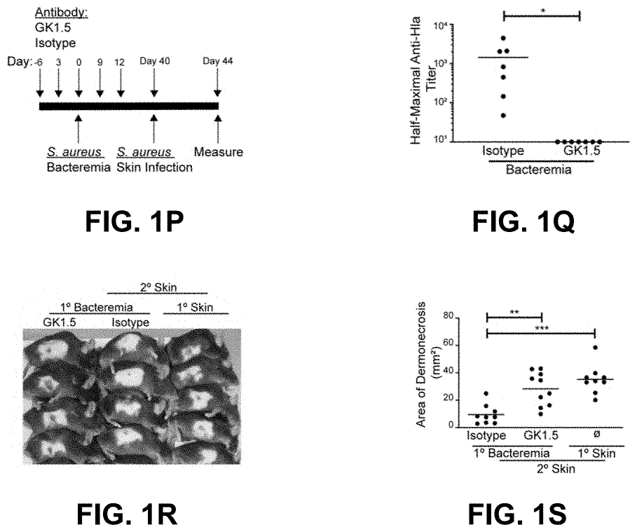

To recapitulate the clinical observation that tissue-specific responses to S. aureus shape immunity, mice were challenged with S. aureus USA300/LAC via intravenous or subcutaneous routes to model bacteremia and skin infection (FIG. 1A). Bacteremic mice experienced approximately 15%-20% weight loss, regaining weight over 12 to 14 days (FIG. 1E), whereas mice exposed to skin infection harbored lesions that peaked within 2 days and resolved by day 14 (FIG. 1F). Mice were challenged with S. aureus skin infection on day 40, and assessed bacterial control 4 days later. Mice challenged with intravenous S. aureus USA300/LAC exhibited smaller lesions during the secondary skin challenge than did mice exposed to a primary skin infection, as evidenced by tissue pathology (FIG. 1B), dermonecrosis (FIG. 1C), and bacterial burden (FIG. 1D). Given the role of S. aureus α-toxin (Hla) in primary and recurrent skin infection, the anti-Hla response was evaluated and found that bacteremia elicited higher IgG levels against Hla (FIG. 1C) and staphylococcal lysates (FIG. 1H) compared with primary skin infection. Following the secondary infection, initial skin exposure resulted in a limited anti-Hla response, as previously observed, relative to the response in mice initially subjected to bacteremia (FIG. 1G). In contrast, anti-staphylococcal antibody titers were indistinguishable (FIG. 1H), suggesting specificity of the response. Primary skin infection was associated with increased CD19⁺ B cells in the draining lymph node (dLN) compared with bacteremic infection, whereas splenic B cell recovery was similar (FIG. 1I), suggesting that the reduced antibody response following primary skin infection was not solely attributable to a quantitative B cell defect. To further evaluate the importance of the antibody response, primary intravenous challenge of mice deficient in mature B cells (μMT) revealed poor control of dermonecrosis (FIGS. 1J and 1K) and bacterial burden (FIG. 1L) during secondary skin infection compared with WT mice, a finding that correlated with loss of the anti-Hla response (FIG. 1M). Delivery of an identical inoculum (5×10⁶) for primary skin and bacteremic infection recapitulated these findings (FIGS. 1N and 1O). Although the clinical and pathologic endpoints of bacteremia and skin infection differ, these models reflect the observation in humans that S. aureus immunity depends on the initial infection site. Therefore these models were viewed as comparative tools to interrogate host immunity.

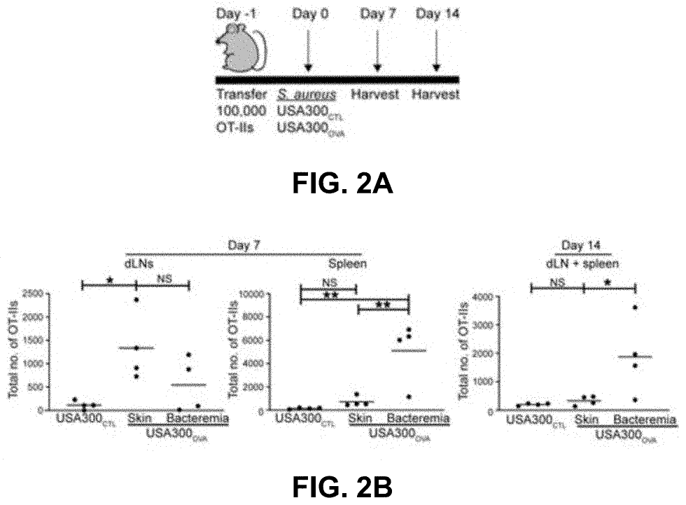

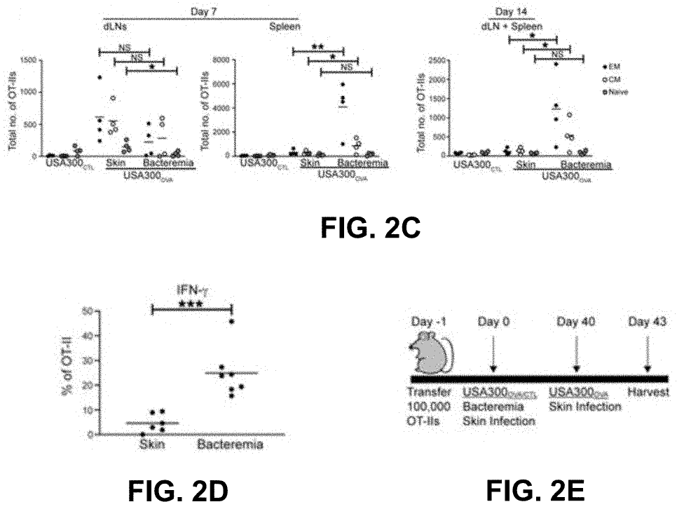

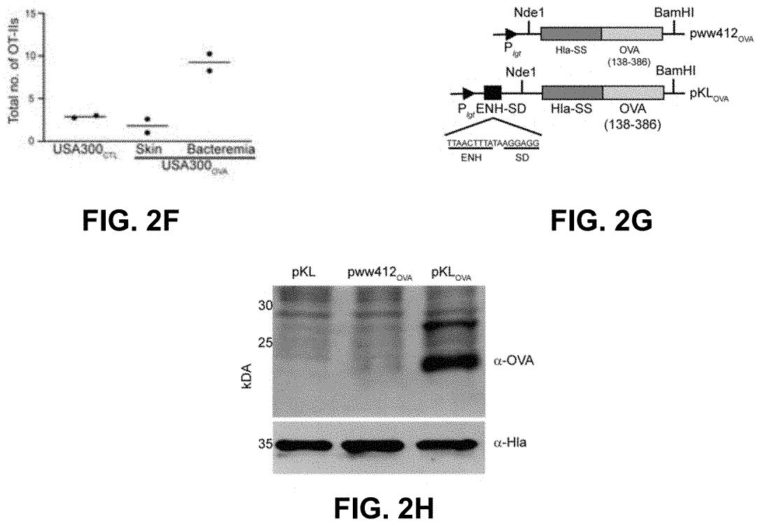

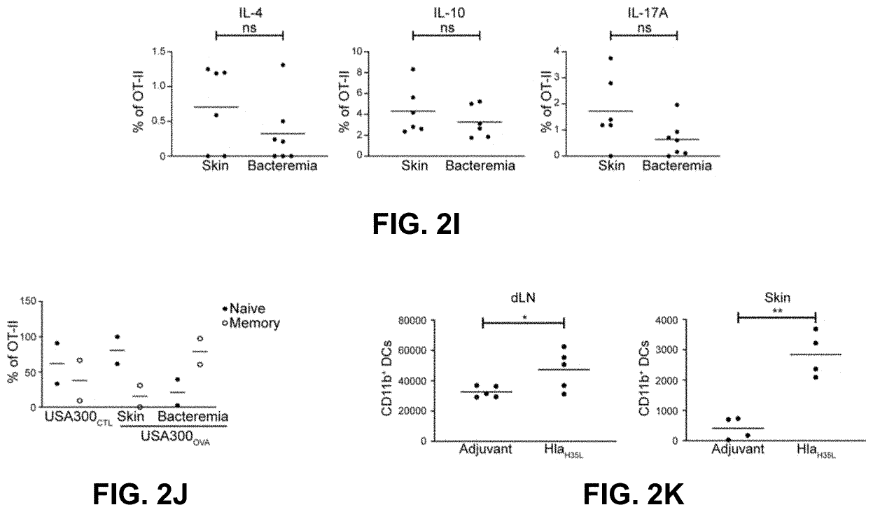

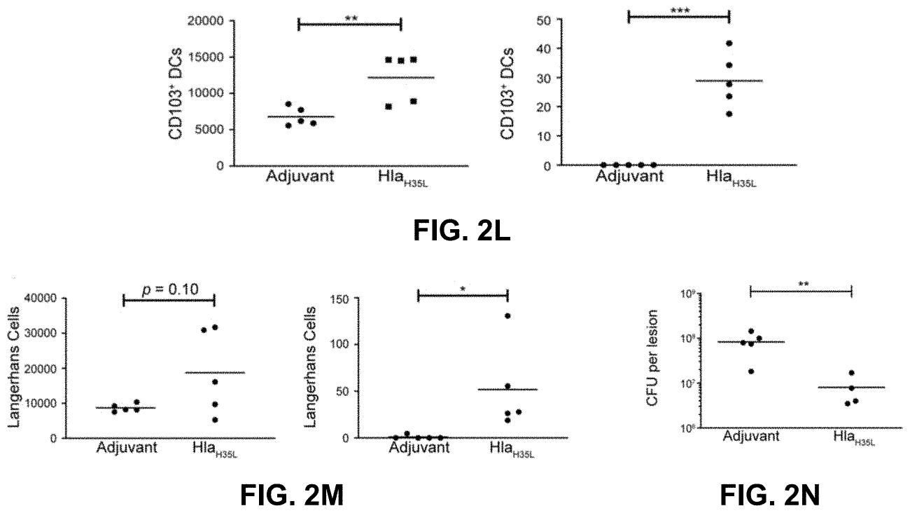

It was hypothesized that the αβ-T cell response may underlie tissue-specific features of immunity. Consistent with this, antibody-mediated depletion of CD4⁺ T cells concurrent with primary bacteremia (FIG. 1P) abrogated the anti-Hla response (FIG. 1Q) and eliminated secondary infection protection (FIGS. 1R and 1S). Although multiple studies have analyzed cellular injury during skin infection, these studies have not evaluated antigen-specific T cell responses in vivo. To this end, the OT-II CD4⁺ T cell system was used in combination with OVA-expressing S. aureus USA300/LAC (USA300_{OVA}). OVA was not detectable when expressed via the Igt promoter (pww412OVA) (FIGS. 2E and 2F), however, expression was achieved using a modified promoter-enhancer element (pKLOVA). To track antigen-specific CD4⁺ T cell responses, CD45.1⁺ OT-II T cells were transferred into mice prior to intravenous or skin challenge with USA300_{OVA} or an empty vector-harboring control strain (USA300_{CTL}) (FIG. 2A). By postinfection day 7, it was observed that OT-II T cells accumulated in the skin dLNs of mice following primary infection via both routes (FIG. 2B, left), whereas only bacteremia prompted splenic OT-II T cell accumulation (FIG. 2B, middle). OVA-specific T cell recovery following skin infection decreased to baseline 14 days after challenge, whereas an approximately 10-fold increase in OT-II T cells persisted after intravenous infection. To assess whether primary infection produced antigen-specific memory T cells, the expression of central memory CD44hiCD62Lhi (CM) and effector memory CD44hiCD62Llo (EM) cell markers were analyzed. Both infection routes induced EM and CM responses on day 7 (FIG. 2C), with an EM predominance in the spleen after bacteremia. Only primary bacteremia elicited a detectable memory OT-II T cell response until day 14.

T cell differentiation toward effector and memory cell phenotypes during infection is shaped by local cues from antigen-presenting cells and the cytokine milieu. Although an IL-17-predominant response to S. aureus infection correlates with epithelial protection, an IFN-γ-dominant T cell response is elicited by systemic infection and required for protection. Our model enables assessment of the cytokine response in antigen-specific T cells and quantification of the memory response. Evaluation of OVA-specific T cells elicited by primary bacteremia revealed an increased percentage of IFN-γ-producing cells (FIG. 2D); the IL-4, IL-10, and IL-17 responses did not distinguish tissue sites (FIG. 2G). To assess the recall response, mice received USA300_{OVA} intravenously or intradermally and were then rechallenged on day 40 to generate skin infection (FIG. 2E). Bacteremia elicited an increase of approximately 3-fold in OT-II T cell accumulation 3 days after rechallenge relative to that seen with skin infection (FIG. 2F), with a divergent trend toward CD44hi memory T cell accumulation (FIG. 2H).

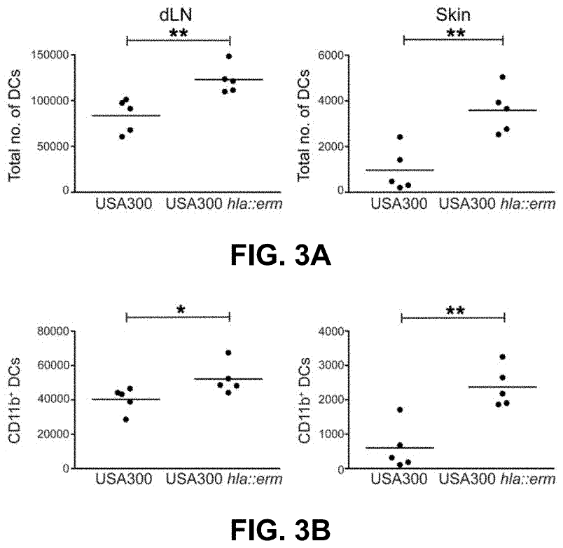

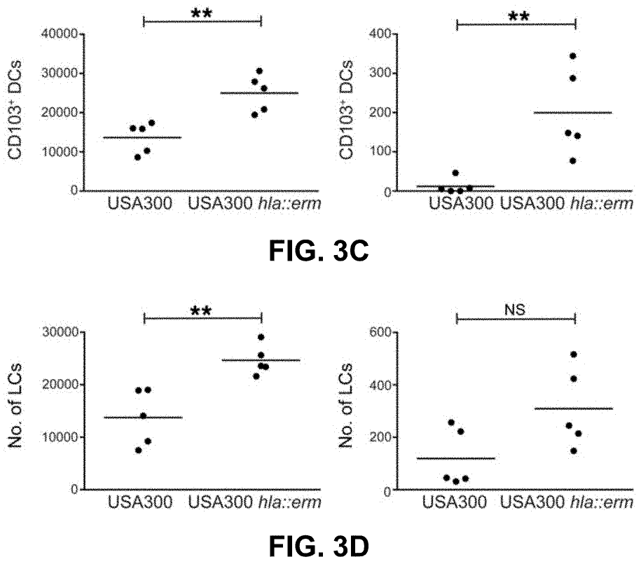

Primary skin infection with an isogenic Hla mutant (USA300 hla::erm) protects against skin rechallenge. It was hypothesized that Hla may modulate DC-T cell crosstalk during primary infection, as CD11b⁺ and CD103⁺ dermal DCs and epidermal Langerhans cells (LCs) are principal skin antigen-presenting cells (26). Mice were subjected to USA300 or USA300 hla::erm skin infection, and evaluated DC numbers 4 days after infection. Administration of USA300 led to a reduction in the dLN and skin DC populations, which were restored in the absence of Hla (FIG. 3A). Analysis of specific cell subpopulations revealed preservation of CD11b⁺ (FIG. 3B) and CD103⁺ (FIG. 3C) DCs in USA300 hla::erm-infected mice. LC numbers showed a trend consistent with protection following USA300 hla::erm infection, with a significant increase in the dLNs (FIG. 3D). Diminution of the DC compartment may be the result of direct cytotoxicity by Hla or other S. aureus toxins, or may reflect tissue injury, in which local cellular damage engenders a microenvironment that is unfavorable for DC survival.

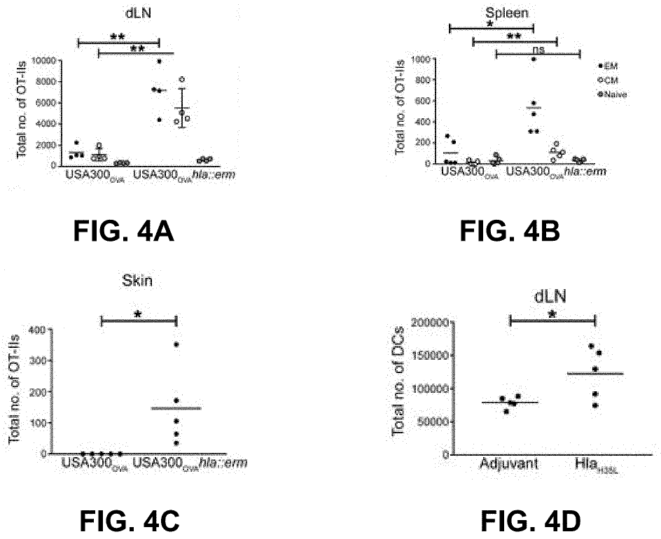

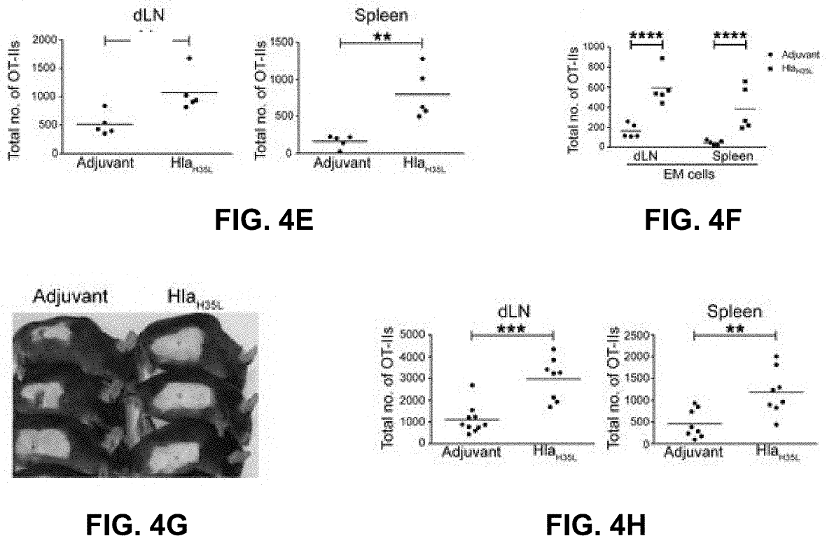

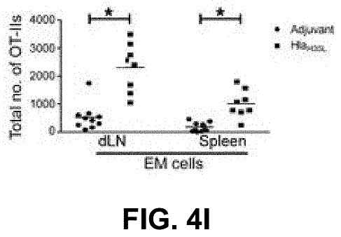

To evaluate the impact of Hla on memory T cell induction, OT-II T cell recipients were infected via the subcutaneous route with USA300_{OVA} or USA300_{OVA} hla::erm, and OT-II T cell accumulation was assessed 7 days after infection. Although these conditions only elicited a minimal anti-OVA IgG response (OD450: USA300_{OVA}, 0.07±0.01; USA300_{OVA} hla::erm, 0.06±0.03 vs. OVA-immunized control 0.8±0.01), Hla deletion augmented EM and CM cell numbers in skin dLNs (FIG. 4A) and spleen (FIG. 4B). OT-II T cells were recovered from the skin only during infection with USA300 OVA hla::erm (FIG. 4C). Together, these data demonstrate that Hla impairs the memory response, thereby limiting antigen-specific T cell localization.

Next, whether active immunization against Hla would protect the cellular immune response during skin infection was examined. Mice were immunized with adjuvant or inactive Hla (HlaH35L) prior to skin challenge. DCs were protected at the dLN and skin sites after infection (FIG. 4D), with accumulation of DCs and LCs observed in HlaH35L-immunized mice (FIGS. 2I, 2J, and 2K). It was found that DC compartment preservation was associated with enhanced generation of antigen-specific T cells (FIG. 4E) that exhibited an effector phenotype (FIG. 4F). S. aureus recovery was reduced in skin lesions of immunized mice (FIG. 2L), mirroring findings upon infection with an Hla-deficient strain. S. aureus expresses leukocidins and phenol-soluble modulins that target DCs, inhibiting antigen uptake, presentation, and T cell proliferation. As species-specific cellular receptors define the activity of multiple staphylococcal toxins, our observations of the role of Hla in modulating the murine antigen-specific T cell response suggest that this response may also be modified in humans through the combined cellular action of toxins. S. aureus may thus rely on multiple virulence factors in skin infection to simultaneously cause injury and manipulate host immunity.

S. aureus colonizes up to 50% of infants by 8 weeks of age, raising the possibility that immunity is templated early in life. Indeed, studies in a Staphylococcus epidermidis neonatal skin colonization model using an antigen-specific reporter T cell system revealed that early antigen exposure promotes immunologic tolerance, characterized by the establishment of commensal-specific Tregs. To determine whether Hla neutralization is sufficient to protect the T cell compartment using a strategy suited for early life intervention, the impact of maternal immunization was evaluated. Mice born to HlaH35L-immunized dams had protection against skin infection relative to the offspring of control-immunized dams (FIG. 4G). Enhanced OT-II T cell recovery (FIG. 4H) characterized by an increase in EM cells was seen in the offspring of HlaH35L-immunized dams (FIG. 4I), suggesting that passive transfer of maternally derived Hla-neutralizing antibodies engenders protection of the antigen-specific T cell response.

This Example underscores the importance of T cell-mediated immunity in protection against S. aureus disease. The T cell response will not only reflect the tissue environment during primary infection, but modulate the B cell-derived humoral response. Therefore, a detailed understanding of T cell specificity and effector phenotype will be beneficial to elicit vaccine-derived protective immunity. If the effects of Hla on T cell-mediated immunity occur during initial skin exposure in humans, the T cell repertoire may be perturbed by colonization or infection in infancy. This consideration has 3 important implications: first, individuals with S. aureus exposure harbor a preexisting T cell repertoire influenced by the pathogen. Thus, post-exposure vaccine trials may not be capable of favorably altering the diversity of the T cell response or specific effector functions necessary for protective immunity. Second, strategies such as maternal immunization and/or infant vaccination may be required to generate population-level protective immunity. Third, the strategic design of vaccine adjuvant and tissue delivery systems will be essential to instruct the T cell effector and memory response. By removing the suppressive effects of Hla on host immune function, immunization against Hla may expand antigen-specific T cell diversity and allow natural S. aureus exposure to amplify the T cell repertoire rather than elicit tolerogenic or suppressive responses.

### Example 2: Development of a S. aureus Vaccine Targeting α-Toxin (Hla)

Staphylococcus aureus is one of the most pressing infectious disease threats that impacts humans worldwide. S. aureus can infect any tissue in the human body. The most common forms of disease include skin infection, pneumonia, bloodstream infection and sepsis, and infection of the muscles, bones, and joints. Owing to its demonstrated ability to rapidly acquire drug resistance, S. aureus was defined by the CDC and WHO as a priority pathogen in urgent need of new strategies for prevention and treatment. Infants and children are frequently colonized with S. aureus even within the first week of life, thus are exposed to staphylococcal antigens that can serve as immunogens. Given the multiple strategies that S. aureus utilizes to subvert the development of protective immunity, the initial exposure to S. aureus early in life may potentiate the development of nonprotective immune responses. When delivered after S. aureus exposure, it is quite possible that vaccines designed to elicit active immunity against staphylococcal antigens will only serve to enhance existing nonproductive responses.

S. aureus α-toxin (Hla) is a small pore-forming cytotoxin produced by almost all clinically relevant strains of this microbe. Hla binds to A Disintegrin and Metalloprotease 10 (ADAM10) on the host cell surface, utilizing this protein as a toxin receptor. ADAM10 binding enables the assembly a homo-heptamer on the membrane, which is a requisite intermediate for the extension of the stem domain of the toxin through the membrane as a classic beta-barrel pore structure. While Hla is not required for S. aureus survival, this toxin is essential for pathogenesis in animal models of severe skin infection, pneumonia, sepsis, peritonitis, corneal infection, and central nervous system infection. Both active and passive immunization targeting Hla provides protection against skin infection, sepsis, pneumonia, and peritonitis in animal models of disease. Several distinct Hla immunogens have been evaluated, including the single point mutation HlaH35L that renders the protein non-toxigenic. Human data exists for the relative role of anti-Hla antibodies as a correlate of protection. The risk of sepsis in adult patients was lower in individuals having higher levels of serum antibody to S. aureus toxins, one of which was Hla. In a more focused study, children experiencing recurrent S. aureus infection in the 12-month period following an initial clinical infection exhibited lower anti-Hla serum titers than children who did not suffer from recurrent infection. Suggesting that S. aureus skin infection dampens the antigen-specific T cell response dependent on the action of Hla. Hla exposure is associated with both quantitative loss of antigen-specific T cells and qualitative alteration of the nature of the effector memory response to infection. Genetic deletion of Hla as well as immunization of mice with the non-toxigenic HlaH35L variant prior to the time of initial infection restores the antigen-specific T cell response and is associated with protection against disease. As S. aureus and Hla exposure may occur in the first days of life, as described herein maternal immunization targeting Hla engenders protection of the T cell response following infection in offspring.

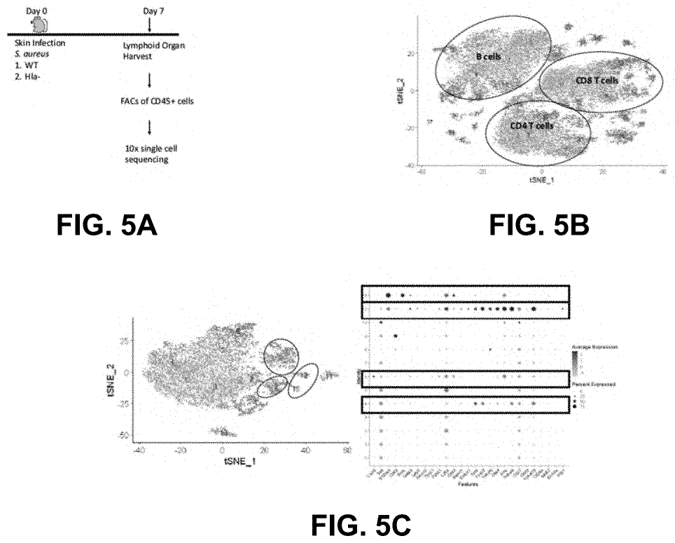

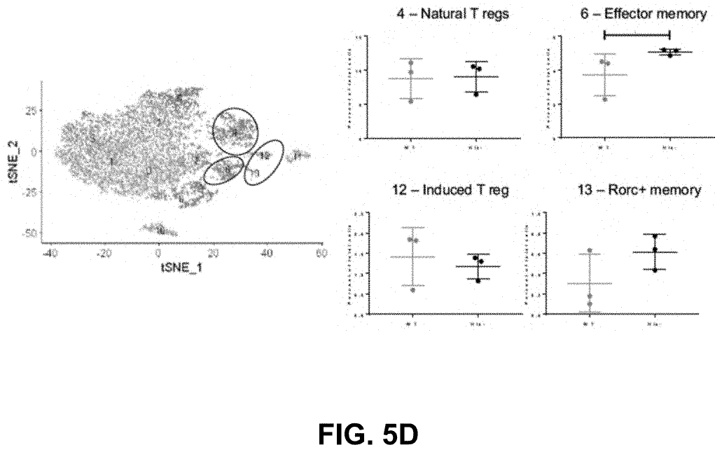

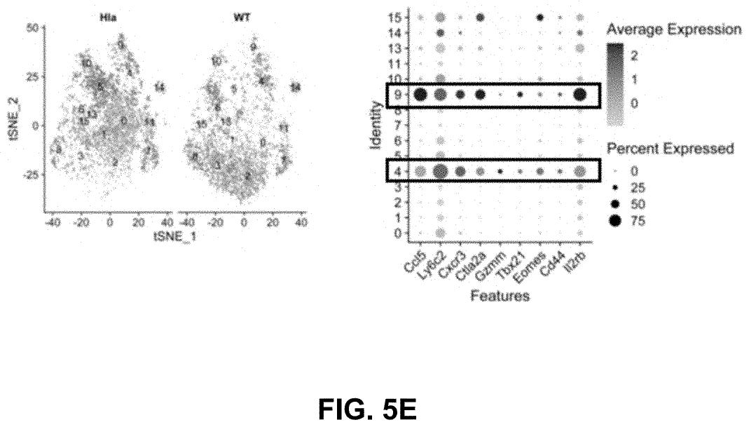

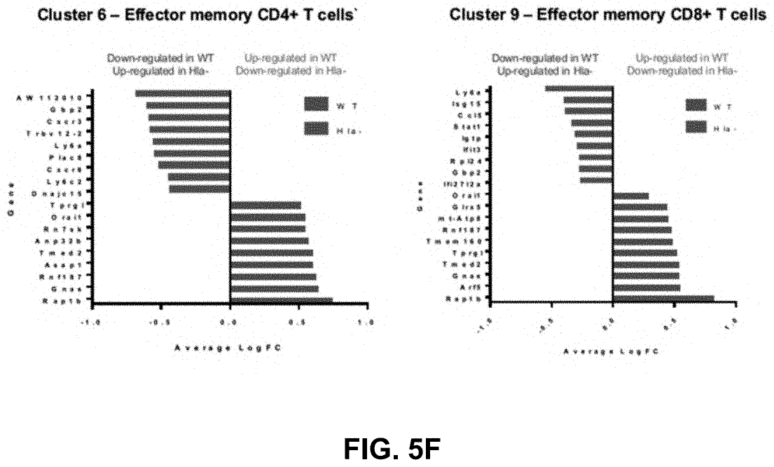

Protection of the T cell response from the effects of Hla is associated with the generation of an effector T cell response characterized by a gene profile in which the chemokines Cxcr3 and Cxcr6 together with the long noncoding RNA AW112010 are upregulated, consistent with patterning of a canonical Th1-skewed T cell response. This response was mapped to individual populations of T cells that are phenotypically distinguished based on their transcriptional profiles (see, e.g., FIG. 5).

Infection and isolation of CD45 positive cells. 4-weeks old male C57BL/6 mice were subcutaneously infected with 1×10⁸ CFU of either wildtype or ΔHla (erm::hla) USA300 strain in 50 μl PBS. Lymph nodes were harvested from infected mice at day 7 post-infection and homogenized to obtain single cell suspension. Contaminating red blood cells were lysed using an ammonium chloride lysis solution. Cells were then stained with DRAQ5 and DAPI (for exclusion of dead cells and cell debris), CD16/32 (FC block) and anti-mouse CD45 antibodies. CD45 positive cells were FACs sorted using the BD FACSAria Fusion cell sorter. Sorted cell single cells were sequenced at the McDonnell Genome Institute (MGI) at Washington University in St. Louis using the Chromium Single Cell system (10×genomic).

Processing of scRNA-seq data. Count matrices produced using Cell Ranger were analyzed in the R statistical working environment (version 3.6.1). Quality control, unsupervised cell clustering and differential gene expression analysis were performed using the Seurat Package (version 4.0.0) in R) as described (https://satijalab.org/seurat/v3.2/pbmc3k_tutorial.html).

The unique transcriptional profiles observed in the presence of Hla suggest that the toxin dampens the generation of a Th1-skewed response and blunts the generation of an effector memory response. This profiling will not only inform further studies of the T cell response to infection, but is also expected to form the basis for defined ‘biomarker-based’ analysis of the T cell response induced by natural infection and in the context of vaccine mediated protection, and thus should enable vaccine targeting.

This observation forms the intellectual basis for pursuing strategic immunization targeting Hla prior to initial exposure to S. aureus in order to protect the development and maturation of the antigen-specific T cell response. As described herein, two distinct approaches are proposed to achieve this goal: 1) active immunization of newborns at the time of birth followed by booster immunizations during the primary series in infancy and early childhood, or 2) maternal immunization to elicit transplacental transfer of anti-Hla neutralizing antibodies for neonatal protection, coupled with active immunization during the primary series in infancy and early childhood.

The present Example shows a single antigen preparation designed to enable population scale, safe and effective immunization targeting Hla. Five exemplary formulations of genetically detoxified purified protein vaccine were generated: 1) HlaH35L in which substitution of amino acid 35 abrogates functional pore formation by destabilizing the heptameric structure, 2) HlaI5A/I7A in which substitution of amino acids 5 and 7 perturb the structure of the amino-latch of Hla, modifying the conformation of the monomer and the ability of the amino latch to contribute to stabilization of the oligomeric pore, 3) HlaD45A/Y118F in which substitution of amino acids 45 and 118 preclude the interaction of the folded prestem domain with the cap domain, thus predicted to alter the structure and receptor binding properties of the monomeric form of Hla, 4) HlaR66A/E70A in which substitution of amino acids 66 and 70 alters the binding properties of the toxin with the host receptor and cell membrane, and 5) HlaDY118-V140 in which native residues Y118-V140 are replaced with an engineered peptide encompassing the predicted T cell epitope KKVFYSFIDDKNHNK (HlaK36-K50)(amino acids 1-15 of SEQ ID NO: 10) flanked by two linker sequences (GPGPG)(SEQ ID NO: 6). As this engineered variant replaces the native stem domain of Hla, the toxicity of the variant is eliminated. It is possible that combinations of these variants would also be favorable for use as vaccine immunogens.

Some antigens below contain repeating sub-units of Hla linked together by an amino acid linker. Sub-unit peptides were picked excluding the signal sequence of the protein. GPGPG (show as underlined below; SEQ ID NO: 6) linkers play dual roles: preventing the generation of junctional epitopes and facilitating the immunoprocessing and presentation of antigen.

Exemplary Hla antigen 1: Repeating units of 50-mer: Generated using the first 50 amino acids (1-50 of SEQ ID NO: 1). Antibodies targeting this region of the protein has been shown to be neutralizing:

(SEQ ID NO: 7)
ADSDINIKTGTTDIGSNTTVKTGDLVTYDKENGMHKKVFYSFIDDKNHN
KGPGPGADSDINIKTGTTDIGSNTTVKTGDLVTYDKENGMHKKVFYSFI
DDKNHNKGPGPGADSDINIKTGTTDIGSNTTVKTGDLVTYDKENGMHKK
VFYSFIDDKNHNKGPGPGADSDINIKTGTTDIGSNTTVKTGDLVTYDKE
NGMHKKVFYSFIDDKNHNKGPGPGADSDINIKTGTTDIGSNTTVKTGDL
VTYDKENGMHKKVFYSFIDDKNHNK.

Exemplary Hla antigen 2: Predicted CD4⁺ T cell-specific epitopes.

(SEQ ID NO: 8)
KKVFYSFIDDKNHNKGPGPGKKVFYSFIDDKNHNKGPGPGKKVFYSFID
DKNHNKGPGPGKKVFYSFIDDKNHNKGPGPGKKVFYSFIDDKNHNK.

Exemplary Hla antigen 3.

(SEQ ID NO: 9)
KKVFYSFIDDKNHNKGPGPGKLLVIRTKGTIAGQYGPGPGTDKKVGWKV
IFNNMVGPGPGGWKVIFNNMVNQNWGGPGPGYGNQLFMKTRNGSMK.

Exemplary Hla antigen 4:

(SEQ ID NO: 10)
KKVFYSFIDDKNHNKGPGPGTDKKVGWKVIFNNMVGPGPGKLLVIRTK
GTIAGQY.

Exemplary Hla antigen 5.

(SEQ ID NO: 11)
TDKKVGWKVIFNNMVGPGPGTDKKVGWKVIFNNMVGPGPGTDKKVGWKV
IFNNMVGPGPGTDKKVGWKVIFNNMVGPGPGTDKKVGWKVIFNNMV

Exemplary Hla antigen 6: Amino latch (amino acids 1-20 of SEQ ID NO:1) peptide variants in the context of the H35L mutant including a combination of single or multiple amino acid substitutions within the first 20 amino acids.

Exemplary Hla antigen 7: Removal of amino latch trypsin sensitivity, e.g., HlaK8A variant and Hla molecules comprising the same.

Exemplary Hla antigen 8: Perturbation of amino latch hydrophobic contacts, e.g., HlaI5A/I7A variant and Hla molecules comprising the same.

Exemplary Hla antigen 9: Perturbation of ADAM10 binding mutant, e.g., HlaR66A/E70A variant and Hla molecules comprising the same.

Exemplary Hla antigen 10: Disengagement of the pre-stem variant, predicted to expose amino latch, e.g., HlaD45A/Y118F variant and Hla molecules comprising the same.

Exemplary Hla antigen 11: Stem deletion variant, e.g., HlaΔK110-Y148 with insertion of amino acid linker following residue 109 and Hla molecules comprising the same.

Exemplary Hla antigen 12: Membrane insertion deletion variant, e.g., HlaΔY118-V140 with insertion of amino acid linker following residue 117 and Hla molecules comprising the same.

Exemplary Hla antigen 13: Protomer-protomer interface variants, e.g., HlaH35 variant in which histidine residue is substituted by any other amino acid and Hla molecules comprising the same.

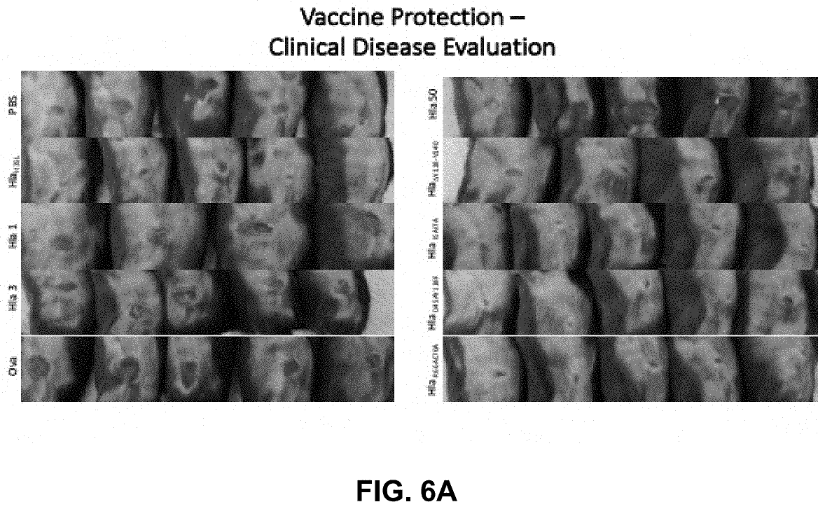

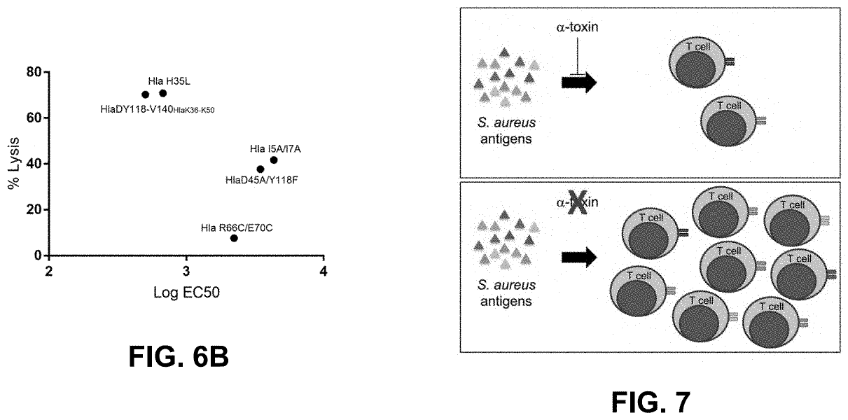

As demonstrated herein, vaccination targeting HlaH35L elicits protection against clinical disease and enables maturation of antigen-specific T cell memory (Example 1). Similarly, as shown below, HlaI5A/I7A, HlaD45A/Y118F, HlaR66A/E70A, and HlaDY118-V140 variants engender protection from primary and recurrent clinical disease (see, e.g., FIG. 6). Additional studies characterize these variants and prioritize their functional properties as a vaccine immunogen including protection of the antigen-specific T cell response to S. aureus, analysis of the phenotypic T cell profile through transcript and expression level profiling, quantification and characterization of the anti-Hla neutralizing antibody response, structure-function correlation of Hla candidate variants with elicited host immune response

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method to reduce or prevent tolerogenic or suppressive T-cell responses or a method to induce a protective T-cell response, in a subject to a bacterial pathogen, the method comprising; administering to the subject a composition comprising a bacterial antigen specific to the bacterial pathogen prior to the subjects first exposure to the bacterial pathogen.

2. The method of claim 1, wherein the composition is administered to a mother of the subject while the subject is in utero.

3. (canceled)

4. The method of claim 1, wherein the composition is administered-to the subject one or more times (a) at birth; (b) within about 1, 2, 3, 4, 5, 10, 15, 20, 25, 30, 35, 40, 45, 50, 55 minutes after birth; (c) within about 1, 2, 3, 4, 5, 6, 7, 8, 9, 10, 11, 12, 13, 14, 15, 16, 17, 18, 19, 20, 21, 22, 23, 24 hours after birth, (d) within about 1, 2, 3, 4, 5, 6, 7 days after birth, or (e) about 1, 2, 3, 4, 5, 6, 7, or 8 weeks after birth.

5-8. (canceled)

9. The method according to claim 1, wherein the bacterial antigen is an attenuated bacterial toxin.

10. The method according to claim 9, wherein the bacterial antigen is a-hemolysin (Hla).

11. The method according to claim 10, wherein the Hla is a modified Hla comprising:
(a) a substitution of a histidine amino acid at position 35 relative to SEQ ID NO: 1 wherein position 35 is substituted with any other amino acid thereby abrogating functional pore formation by destabilizing the heptameric structure;
(b) a modified Hla comprising a combination of single or multiple amino acid substitutions within the first 20 amino acids relative to SEQ ID NO: 1;
(c) a substitution of amino acids at position 45 and 118 relative to SEQ ID NO: 1, thereby precluding the interaction of the folded prestem domain with the cap domain, thus altering the structure and receptor binding properties of Hla;
(d) a substitution of amino acids at position 66 and 70 relative to SEQ ID NO: 1 thereby altering the binding properties of the toxin with the host receptor and cell membrane;
(e) a substitution of amino acids Y118-V140 KKVFYSFIDDKNHNK (amino acids 1-15 of SEQ ID NO: 10) flanked by two linker sequences (GPGPG)(SEQ ID NO: 6);
(f) comprising a substitution of amino acid at position 8 relative to SEQ ID NO:1; or
(g) comprises the amino acid sequence of SEQ ID NO: 7, SEQ ID NO: 8, SEQ ID NO: 9, SEQ ID NO: 10, or SEQ ID NO: 11.

12-23. (canceled)

24. The method according to claim 1, wherein the bacterial pathogen is Staphylococcus aureus.

25. A method to elicit an immune response to Staphylococcus aureus, or treat an infection by Staphylococcus aureus, or prevent an infection by Staphylococcus aureus, in a subject in need thereof, the method comprising; administering to the subject a composition comprising a modified Hla prior to the subjects first exposure to the Staphylococcus aureus.

26. The method of claim 25, wherein the composition is administered to a mother of the subject while the subject is in utero.

27. (canceled)

28. The method of claim 1, wherein the composition is administered-to the subject one or more times (a) at birth; (b) within about 1, 2, 3, 4, 5, 10, 15, 20, 25, 30, 35, 40, 45, 50, 55 minutes after birth; (c) within about 1, 2, 3, 4, 5, 6, 7, 8, 9, 10, 11, 12, 13, 14, 15, 16, 17, 18, 19, 20, 21, 22, 23, 24 hours after birth, (d) within about 1, 2, 3, 4, 5, 6, 7 days after birth, or (e) about 1, 2, 3, 4, 5, 6, 7, or 8 weeks after birth.

29-34. (canceled)

35. The method according to claim 25, wherein the modified Hla comprises:
(a) a substitution of a histidine amino acid at position 35 relative to SEQ ID NO: 1 wherein position 35 is substituted with any other amino acid thereby abrogating functional pore formation by destabilizing the heptameric structure;
(b) a modified Hla comprising a combination of single or multiple amino acid substitutions within the first 20 amino acids relative to SEQ ID NO: 1;
(c) a substitution of amino acids at position 45 and 118 relative to SEQ ID NO: 1, thereby precluding the interaction of the folded prestem domain with the cap domain, thus altering the structure and receptor binding properties of Hla;
(d) a substitution of amino acids at position 66 and 70 relative to SEQ ID NO: 1 thereby altering the binding properties of the toxin with the host receptor and cell membrane;
(e) a substitution of amino acids Y118-V140 KKVFYSFIDDKNHNK (amino acids 1-15 of SEQ ID NO: 10) flanked by two linker sequences (GPGPG)(SEQ ID NO: 6);
(f) comprising a substitution of amino acid at position 8 relative to SEQ ID NO:1; or
(g) comprises the amino acid sequence of SEQ ID NO: 7, SEQ ID NO: 8, SEQ ID NO: 9, SEQ ID NO: 10, or SEQ ID NO: 11.

36. The method according to claim 25, wherein the modified Hla comprises a combination of single or multiple amino acid substitutions within the first 20 amino acids relative to SEQ ID NO: 1, and a substitution of a histidine amino acid at position 35 relative to SEQ ID NO: 1.

37-41. (canceled)

42. The method of according to claim 25, wherein the modified Hla comprises a substitution of a histidine amino acid at position 35 relative to SEQ ID NO: 1, and a substitution of amino acids at position 66 and 70 relative to SEQ ID NO: 1.

43-70. (canceled)

71. An isolated modified Hla peptide comprising:
(a) a substitution of a histidine amino acid at position 35 relative to SEQ ID NO: 1
(b) a combination of single or multiple amino acid substitutions within the first 20 amino acids relative to SEQ ID NO:1;
(c) a substitution of amino acids at position 5 and 7 relative to SEQ ID NO: 1;
(d) a substitution of amino acids at position 45 and 118 relative to SEQ ID NO: 1;
(e) a substitution of amino acids at position 66 and 70 relative to SEQ ID NO: 1;
(f) a substitution of amino acids Y118-V140 KKVFYSFIDDKNHNK (amino acids 1-15 of SEQ ID NO: 10) flanked by two linker sequences (GPGPG)(SEQ ID NO: 6); or
(g) a substitution of amino acid at position 8 relative to SEQ ID NO: 1.

72. An isolated modified Hla peptide comprising a combination of single or multiple amino acid substitutions within the first 20 amino acids relative to SEQ ID NO:1, and a substitution of a histidine amino acid at position 35 relative to SEQ ID NO:1.

73-77. (canceled)

78. An isolated modified Hla peptide comprising a substitution of a histidine amino acid at position 35 relative to SEQ ID NO:1, and a substitution of amino acids at position 66 and 70 relative to SEQ ID NO: 1.

79-83. (canceled)

84. A nucleic acid encoding the isolated modified Hla according to any one of the claims 71-83.

85. A vector comprising the nucleic acid sequence of claim 84.

86. A pharmaceutical composition comprising the isolated modified Hla of claim 71.
